(19) United States
(12) Patent Application Publication
(10) Pub. No.: US 2024/0132912 A1
(43) Pub. Date: Apr. 25, 2024

(54) CONSTRUCTS AND METHODS FOR DELIVERYING MOLECULES VIA VIRAL VECTORS WITH BLUNTED INNATE IMMUNE RESPONSES

(71) Applicant: NXGEN VECTOR SOLUTIONS, LLC, Washington, DC (US)

(72) Inventor: Joseph E. Rabinowitz, Philadelphia, PA (US)

(21) Appl. No.: 18/366,495

(22) Filed: Aug. 6, 2023

Related U.S. Application Data
(63) Continuation of application No. 17/317,560, filed on May 11, 2021, now abandoned.
Application No. 17/317,560 is a continuation of application No. 15/099,651, filed on Apr. 15, 2016, now Pat. No. 11,015,210.
Application No. 15/099,651 is a continuation of application No. 14/211,666, filed on Mar. 14, 2014, now abandoned.
(60) Provisional application No. 61/785,368, filed on Mar. 14, 2013.

(51) Int. Cl. C12N 15/86; A61K 48/00; C12N 7/00

(52) U.S. Cl. CPC C12N 15/86; A61K 48/0058; A61K 48/0075; A61K 48/0091; C12N 7/00; C07H 21/04; C12N 2750/14141

(57) ABSTRACT

A CpG-modified recombinant adeno-associated viral (AAV) vector is described. The vector carries a nucleic acid molecule comprising AAV inverted terminal repeat (ITR) sequences and an exogenous gene sequence under the control of regulatory sequences which control expression of the gene product, in which the nucleic acid sequences carried by the vector are modified to significantly reduce CpG di-nucleotides such that an immune response to the vector is reduced as compared to the unmodified AAV vector. Also provided are methods and regimens for delivering transgenes using these AAV viral vectors, in which the innate immune response to the vector and/or transgene is significantly modulated.

### CROSS-REFERENCE TO RELATED APPLICATIONS

This application is a continuation application of U.S. application Ser. No. 17/317,560, filed on May 11, 2021, which is a continuation application of U.S. application Ser. No. 15/099,651, now U.S. Pat. No. 11,015,210, filed on Apr. 15, 2016 as a continuation application of U.S. application Ser. No. 14/211,666, filed Mar. 14, 2014, which in turn claims priority from U.S. Provisional Application No. 61/785,368, filed Mar. 14, 2013. The contents of each of the aforementioned applications are incorporated herein by reference in their entireties.

### STATEMENT OF FEDERALLY SPONSORED RESEARCH OR DEVELOPMENT

This invention was made with government support under grant numbers HL091096, HL007954 and AI007324 awarded by the National Institutes of Health (NIH). The government has certain rights in the invention.

### SEQUENCE LISTING

This application contains a sequence listing filed in ST.26 format entitled “140014-1010 Sequence Listing” created on Aug. 7, 2023, and having 43,908 bytes. The content of the sequence listing is incorporated herein in its entirety.

### BACKGROUND OF THE INVENTION

Adeno-associated virus (AAV) is a small, non-enveloped human parvovirus that packages a linear strand of single stranded DNA genome that is 4.7 kb. The capsid of an AAV contains 60 copies (in total) of three viral proteins (VPs), VP1, VP2, and VP3, in a predicted ratio of 1:1:10, arranged with T=1 icosahedral symmetry [H-J Nam, et al., J Virol., 81(22): 12260-12271 (Nov 2007)). The three VPs are translated from the same mRNA, with VP1 containing a unique N-terminal domain in addition to the entire VP2 sequence at its C-terminal region [Nam et al., cited above]. VP2 contains an extra N-terminal sequence in addition to VP3 at its C terminus. In X-ray crystal structures of the AAV2 [Q. Xie, et al., Proc Natl. Acad. Sci. USA 99:10405-10410 (2002)] and AAV4 [L. Govindasamy, et al., J. Virol., 80:11556-11570] capsids and all other structures determined for parvovirus capsids, only the C-terminal polypeptide sequence in the AAV capsid proteins C530 amino acids) is observed. The N-terminal unique region of VP1, the VP1-VP2 overlapping region, and the first 14 to 16 N-terminal residues of VP3 are disordered [L. Govindasamy, et al., and Q. Xie et al., cited above].

Productiveinfection by AAV occurs only in the presence of a helper virus, either adenovirus or herpes virus. In the absence of a helper virus, AAV integrates into a specific point of the host genome (19q 13-qter) at a high frequency, making AAV the only mammalian DNA virus known to be capable of site-specific integration. See, Kotin et at., 1990, PNAS, 87: 2211-2215. However, recombinant AAV, which does not contain any viral genes and only a therapeutic/marker expression cassette packaged in an AAV capsid, does not integrate into the genome. Instead the recombinant viral genome fuses at its ends via inverted terminal repeats to form circular, episomal forms characterized by long term gene expression.

The ability of AAV vectors to achieve long-term expression of the transgene product has been attributed to their relatively low immunogenicity. However, in some experimental settings, attendant immune responses have com promised the outcome of AAV-mediated gene therapy. How AAV activates the innate immune system remains unknown. In a study published by J. Zhu et al, J Clin Invest, Vol 119, No. 8 (August 2009), it is reported that the innate immune recognition of AAV2 by plasmacytoid dendric cells (DC) was mediated by TLR9 and dependent on My088. Activation of the TLR9-MyD88 pathway was independent of the nature of the transgene. Similarly, other serotypes of AAV, such as AAV1 and AAV9, also activated innate immunity through the TLR9-MyD88 pathway. The authors conclude that their observations suggest that strategies to block the TLR9-MyD88-type I IFN pathway may improve the clinical outcome of AAV-mediated gene therapy.

There have been attempts to modulate the innate immune response to AAV vectors, which approaches have involved various methods of disrupting the TLR9-MyD88-Type I IFN signaling pathway. One such approach is described by Y. Yang et al, US Published Patent Application No. 2011/0070241, published Mar. 24, 2011, which describes co-administration of an antagonist of this pathway with the viral vector. Another approach is described in Yew et al, US Published Patent Application No. 2012/0009222, published Jan. 12, 2012, which complexes lipidoids with polynucleotides such as CpG oligonucleotides, in an attempt to modulate innate immune responses. See, also, G. L. Rogers, et al, Frontiers in Microbiology, Vol. 2, Article 194 (September 2011), a review article which describes reducing vector load may be an alternative to artificially blocking the immune response with drugs. The authors of this paper also report on evidence that self-complementary (sc) AAV vectors induce a greater immune response than single-stranded (ss) AAV, indicating that there is some speculation this is perhaps due to a lack of stability of the viral capsid in scAAV vector. Rogers et al, further report that the vector cassette does not affect the response and that it is unlikely specific sequences in the DNA are responsible [Rogers et al, cited above, page 8, spanning columns 1 and 2] for the response.

The approaches taken to date to address the innate imm une responses to AAV have focused on the AAV capsid. In addition to attempts to modulate Toll-like receptor 9 (TLR9) with short-term immunomodulators, e.g., TLR9 antagonists, attempts have been made to make modifications to the capsid, e.g., generating tyrosine mutant AAV capsids for AAV vectors to address these concerns.

What are needed in the art are constructs and methods for AAV-mediated gene delivery that induce reduced or no detectable innate immune responses.

### SUMMARY OF THE INVENTION

In one aspect, an AAV vector having a CpG-reduced or CpG-depleted nucleic acid sequence packaged within an AAV capsid. Because the AAV vector is viral in origin and contains a capsid composed of proteins, no modification to the viral capsid is necessary. A CpG-modified AAV vector of the invention contains AAV inverted terminal repeat (ITR) sequences and an exogenous gene sequence under the control of regulatory sequences that control expression of the gene product. The nucleic acid sequences of one or more, and preferably all, of these elements are modified to reduce CpG di-nucleotides such that an immune response toward the vector and/or transgene is reduced as compared to the unmodified (aka wild type) AAV vector. Suitably, the AAV vector contains no other genomic AAV sequences.

In one embodiment, the transgene sequence contains a reduced number of CpG di-nucleotides as compared to the native coding sequence for the gene product. In another embodiment, the regulatory sequences are mutated to reduce or eliminate CpG di-nucleotides. In still another embodiment, the 5′ and/or 3′ terminal repeat sequences are mutated to reduce or eliminate native CpG di-nucleotides.

In another aspect, the invention provides a composition comprising a CpG-modified DNA vector as described herein and a pharmaceutically acceptable carrier.

In a further aspect, the invention provides a method for improving adeno-associated virus (AAV)-mediated gene expression by generating an AAV viral particle comprising a modified packaging insert, wherein said packaging insert comprises a nucleic acid molecule comprising AAV inverted terminal repeats (ITRs) sequences or functional equivalents thereof, e.g., 5′ AAV ITR and 3′ AAV ITR (which ITRs may be independently selected from CpG-modified or wild-type ITRs and optionally CpG-modified self-complementary ITRs) and an exogenous gene sequence under the control of regulatory sequences which control expression of the gene product, wherein said sequences of said nucleic acid molecule are modified to reduce CpG di-nucleotides such that an immune response to the vector is reduced as compared to the unmodified AAV vector without significant reduction in expression of the gene product; and delivering the AAV to a subject intramuscularly.

In still another aspect, the invention provides a regimen for repeat administration of gene product. The regimen comprises delivering to a subject an AAV vector having an AAV capsid having packaged therein a CpG-modified nucleic acid molecule carrying an exogenous gene sequence and delivering to the subject a second vector comprising the exogenous gene sequence.

Still other aspects and advantages of the invention will be apparent from the following detailed description of the invention.

### DESCRIPTION OF DRAWINGS

FIG. 1 provides representative sections of X-gal histochemical stain of muscle from wild-type (WT) and TLR9 knock-out (KO) mice injected intramuscularly (IM) with 1×10¹¹ genome copies (GC) of AAVrh32.33nLacZ (nuclear LacZ). 4 mice/group.

FIG. 2A is a bar chart comparing nLacZ reactive CD8+ T cells in WT and TLR9KO mice on days 7, day 14, day 21, day 35, day 42 and day 60 following intramuscular injection with 1×10¹¹ GC of AAVrh32.33nLacZ. Results represent the mean+/−SEM of tetramer positive or cytokine-producing cells from at least n=3 per group.

FIG. 2B is a bar chart showing gamma interferon (IFNy) levels for both wild-type and TLR9 knock out mice on days 35 and 60 following intramuscular injection with 1×10¹¹ GC of AAVrh32.33nLacZ. Results represent the mean+/−SEM of cytokine-producingcells that are reactive toward transgene (nLacZ) and AAVrh32.33 capsid antigen from at least n=3 per group. FIG. 3 provides photographs of representative muscle sections (4 mice per group) recovered from WT and TLR9KO mice that received J. M. injection of 1×10¹¹ GC of AAVrh32.33nLacZ. Sections were stained with anti-CD4 and anti-CD8 antibody (Ab) and examined by fluorescent miscroscopy. 4 mice per group.

FIG. 4 shows photographs of X-gal histochemical stains of muscle from WT and TLR9KO mice injected intramuscularly with 1×10¹¹ GC of AAVrh32.33nLacZ. Representative sections are from 35 and 60 days post-injection. 4 mice per group.

FIG. 5 provides photographs of muscle section that was recovered from WT and TLR9KO mice that received I. M. injection of either 1×10¹¹ GC of AAVrh32.33 or AAV8 expressing nLacZ. Sections recovered on days 35 and 60 were stained with anti-MHC II Ab and examined by fluorescent miscroscopy. Representative sections are shown. 4 mice per group.

FIG. 6 provides a series of bar charts providingtranscript levels of various cytokines assessed by quantitative reverse transcriptase (RT) polymerase chain reaction (PCR) following isolation of RNA from the gastric muscle of WT and TLRKO mice administered 1×10¹¹ GC AAVrh32.33nLacZ or AAV8nLacZ at kinetic time points (2, 6, 12, 24 hours). Transcript levels of MIP-1α, MIP-1β, IL-1β, IL-6, MIP-2 and MCP-1 were assessed by quantitative RT-PCR. Results depict the mean of RNA expression. n=4 mice per group.

FIG. 7A illustrates the structure and sequence of the wild-type AAV2 inverted terminal repeat showing AAV2 Rep binding site (RBS), spacer sequence, terminal resolution site (trs), and rep binding element [SEQ ID NO:4]. FIG. 7B illustrates the AAV2 inverted terminal repeat with CpG dinucleotides outside of the RBS deleted [SEQ ID NO:5].

FIG. 8 provides photographs of representative sections of X-gal histoc hemical stains of muscle from WT mice injected J. M. with 1×10¹¹ GC of AAVrh32.33LacZCpG+ or of AAVrh32.33LacZCpG − vectors. Muscle was harvested at day 35 or day 60 post-injection. 4 mice per group.

FIG. 9A is a bar chart illustrating the percentage of LacZ reactive CD8+ T cells in wild-type mice that were injected I. M. with 1×10¹¹ GC of AAVrh32.33LacZCpG+ or CpG− vectors. Lymphocytes from the mice were isolated from whole blood at various time points post-injection, i.e., days 14, 21, 28, 42 and 60. Lymphocytes were subsequently stained using the PE-conjugated H-2K^{b}-ICPMYARV tetramer together with a FITC-conjugated anti-CD8 Ab to determine the percentage of Lael-specific CDS+ T cells in the total CDS+ T cell population. Results represent the mean+/−SEM of tetramer positive cells from at least n=3 recipients per group.

FIGS. 9B-9D are scattergrams illustrating the results of the same experimen t as described in FIG. 9A. FIGS. 9B-9D provide a scattergram which illustrates the results of the study in the mice injected with the AAVrh32.33LacZCpG+ vectors (FIG. 9B), AAVrh32.33LacZCpG−vectors (FIG. 9C) as compared to naive animals (FIG. 9D), and which charts the concentration of the gal tetramer (PE-conjugated H-2K^{b}-ICPMYARV tetramer) versus the concentration CD8+ cells. FIG. 9E is a bar chart showing gamma interferon (IFNy) levels for wild-type mice on days 35 and 60 following int ramuscular injection with 1×10¹¹ GC of AAVrh32.33LacZCpG+ or AAVrh32.33LacZCpG−vectors. Results represent the mean+/−SEM of cytokine-producing cells that are reactive toward transgene (nLacZ) and AAVrh32.33 capsid antigen from at least n=3 per group.

FIG. 10 provides photographs of representative muscle sections harvested from WT mice that received I. M. injection of 1×10¹¹ GC of AAVrh32.33nLacZCpG+ or CpG− vector. Sections from days 35 and 60 following vector administration were stained with anti-CD4 and anti-CDS Ab and examined by fluorescent microscopy.

FIGS. 11A-11E provide the sequences of the wild-type (+CpG)[SEQ ID NO: 6] and CpG-depleted (−CpG) [SEQ ID NO: 7] Lacz coding sequence in an alignment which further provides a consensus sequence.

FIGS. 12A-12G provide the sequences of a CpG-modified human TnT intron with S1001 exons (−CpG) [SEQ ID NO: 8] in an alignment with the wild-type sequence (+CpG) [SEQ ID NO: 8] and a consensus sequence.

FIGS. 13A-13C provide the sequences of the wild-type (+CpG) [SEQ ID NO: 10] and CpG-depleted (−CpG) firefly luciferase [SEQ ID NO: 11] coding sequence in an alignment which further provides a consensus sequence.

FIGS. 14A-14Q provide an alignment containing a CpG modified vector (pTJU28-ssCMV-S100A1+intron-CpG) with CpG depleted ITRs CMV-EFI-alpha enhancer/promoter S100A1 therapeutic transgene interrupted by a human troponin T intron 9 modified to be CpG (−) followed by an atrial natriuretic factor (ANF) 3′UTR and poly A+ modified to be CpG (−) [SEQ ID NO: 12] compared to the synthesized DNA fragment containing the SI00Al+intron+ANF sequence [SEQ ID NO:13] then compared to the human troponin T intron 9 sequence [SEQ ID NO: 14).

FIGS. 15A-15C provides diagrams of three pTJU vectors containing CpG depleted ITRs, CpG (−) transgenes (LacZ, firefly luciferase and human S100A1) and CpG (−) control elements.

FIG. 16 is a chart showing comparable LacZ plasmid expression for CpG+ and CpG-AAV vector constructs. HeLa cells were transfected with CpG+ and CpG-AAV expression plasmids. Four days post transfection cells were assayed for-galactosidase activity using the mammalian-galactosidase assay kit as instructed for adherent cells. Absorbance was measured at 405 nm on a TECAN® Infinite M1000 PRO plate reader.

FIG. 17 is a chart showing significantly reduced Th1 responses in mice that receive RhCpG-vector on day 14 post administration. Splenocytes were harvested 7 and 14 days intramuscular injection of RhCpG+ or RhCpG-vector. ELISPOT was performed to quantify spots of IFN-gamma per million cells. Transgene reactive Th1 responses are similar between RhCpG+ and RhCpG-injected mice on day 7 but significantly reduced in RhCpG-gene transferred mice on day 14.

### DETAILED DESCRIPTION OF THE INVENTION

The present inventors have found that there is a reduction in immune response to a product expressed from a nucleic acid molecule expression cassette carried by an AAV vector in which the expression cassette has been CpG-modified to contain fewer CpG di-nucleotides. This contrasts with previous reports in the literature that AAV expression cassette sequences do not affect innate immune responses and this invention provides constructs and an approach which differs from that previously taken with AAV which involve a focus on the capsid involvement in the immune response.

A CpG-modified adeno-associated viral (AAV) vector is described herein. As used herein, a cytosine monophosphate (C) followed by a guanine monophosphate (G) in a nucleotide sequence is referred to as a CpG dinucleotide. In eukaryotes, the cytosine residues of CpG dinucleotides are often methylated to 5-methyl-cytosine (mCpG). In bacterial genomes, CpG dinucleotides are typically unmethylated. As used throughout this specification, both methylated and unmethylated CpG are encompassed by the use of the term “CpG” or “CpG dinucleotide), unless methylated on unmethylated CpG is specified.

In one embodiment, an AAV vector is a viral particle having an AAV capsid in which a nucleic acid molecule is packaged. In one embodiment, the AAV vector lacks any functional AAV coding sequences, e.g., is deleted in AAV rep and cap coding sequences. Preferably, the rep and cap coding regions are entirely deleted. Alternatively, these regions are at least functionally deleted (i.e., incapable of producing rep or cap proteins). To the extent any portion of the coding sequences are retained in the AAV vector, they are CpG-depleted and, preferably, CpG-free. Suitably, the nucleic acid molecule contains sequences exogenous to the AAV capsid source, including a nucleic acid sequence which encodes a desired product for delivery to a selected host cell and regulatory sequences which direct expression thereof. The nucleic acid molecule also contains two AAV ITRs, located 5′ and 3′ to the coding sequences, or functional equivalents to these AAV ITRs. These AAV ITRs may be from the same source as the capsid, or may be from a different AAV from the capsid which permits replication of the expression cassette and packaging into the viral particle. Two AAV ITRs in a single vector may also be from different sources from each other. Where the AAV ITRs are from a different source than the AAV capsid, the resulting viral vector particle is termed a pseudotyped AAV.

As previously described, the present invention does not require any modifications to the AAV capsid or to the capsid coding sequence in the plasmid utilized for production of the AAV viral vector particle. Rather, it is the sequence carried within the AAV capsid which is modified to reduce CpG di-nucleotides such that the immune response is reduced following delivery of the vector as compared to the response generated by the unmodified AAV vector.

As used herein, the phrase “CpG-reduced” or “CpG-depleted” refers to a nucleic acid sequence which is generated, either synthetically or by mutation of a nucleic acid sequence, such that a majority of the CpG di-nucleotides are removed from the nucleic acid sequence. In some instances, all CpG motifs are removed to provide what is termed herein modified CpG-free sequences. The CpG motifs are suitably reduced or eliminated not just in a coding sequence (e.g., a transgene), but also in the non-coding sequences, including, e.g., 5′ and 3′ untranslated regions (UTRs), promoter, enhancer, polyA, ITRs, introns, and any other sequences present in the nucleic acid molecule. For the coding sequence, the DNA (5′ to 3′ direction) and codon triplets must be modified as well as the interface between triplets.

For example, CpG di-nucleotides may be located within a codon triplet for a selected amino acid. In one embodiment, the CpG di-nucleotides allocated within a codon triplet for a selected amino acid is changed to a codon triplet for the same amino acid lacking a CpG di-nucleotide.

| | DNA Triplets | |
|---|---|---|
| | Containing | DNA Triplets |
| Amino Acid | CpG | Lacking CpG |
| Serine (Ser or S) | TCG | TCT, TCC, TCA, |
| | | AGT, AGC |
| Proline (Pro or P) | CCG | CCT, CCC, CCA |
| Threonine (Thr or T) | ACG | ACA, ACT, ACC |
| Alanine (Ala or A) | GCG | GCTGCC, GCA |
| Arginine (Arg or R) | CGT, CGC, | AGAAGG |
| | CGA, CGG | |
| Aspararune (Asn, N) | | AAT, AAC |
| Aspartic acid (Asp, | | GAT, GAC |
| D) | | |
| Cvsteine (Cvs, C) | | TGT, TGC |
| Glutamic acid (Glu, | | GAA, GAG |
| E) | | |
| Glutamine (Gin, Q) | | CAA, CAG |
| Glycine (Gly, G) | | GGT, GGC, GGA, |
| | | GGG |
| Histidine (His, H) | | CAT, CAC |
| Isoleucine (Ile D | | ATT, ATC, ATA |
| Leucine (Leu, L) | | CTT, CTC, CTA, |
| | | CTG, TTA, TTG |
| Lvsine <Lvs, K) | | AAA, AAG |
| Methionine (Met, M) | | ATG |
| Phenylalanine (Phe, | | TTT, TTC |
| F) | | |
| stop | | TAA, TAG, TGA |
| Trvotoohan (Tm, W) | | TGG |
| Tyrosine (Tvr, Y) | | TAT, TAC |
| Valine (Val, V) | | GTT, GTC, GTA, |
| | | GTG |

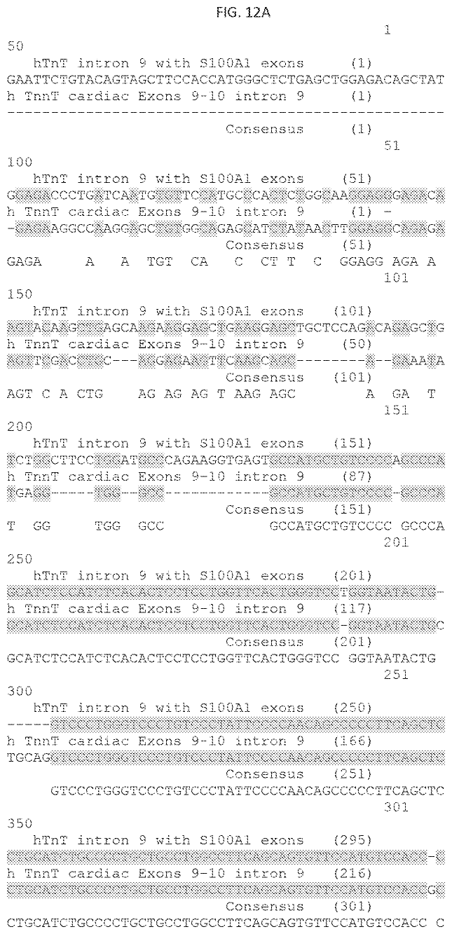

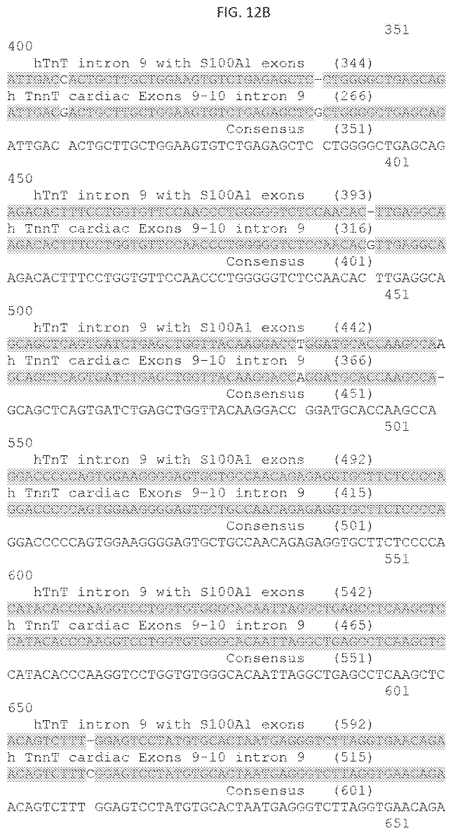

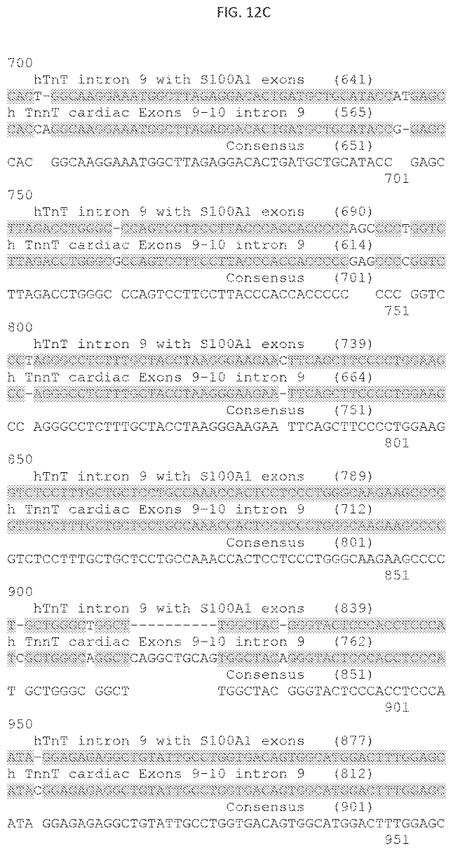

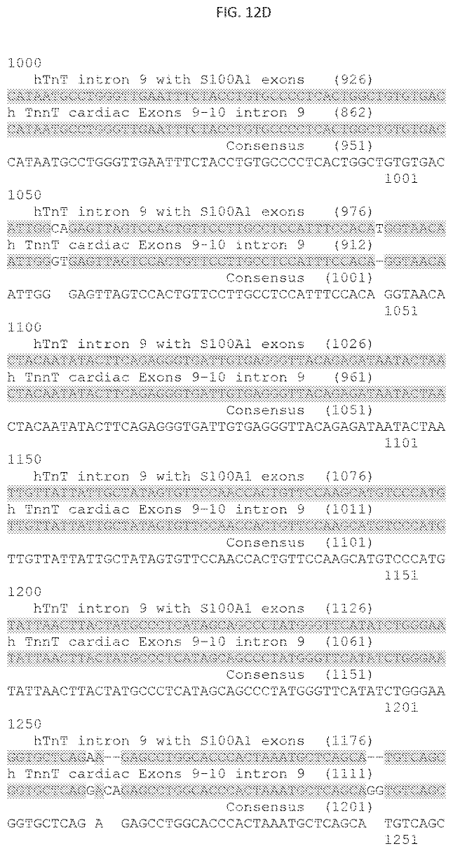

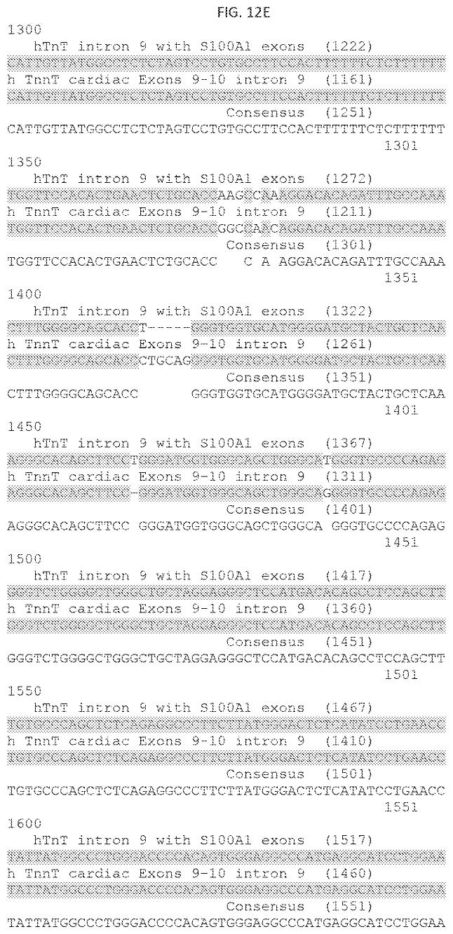

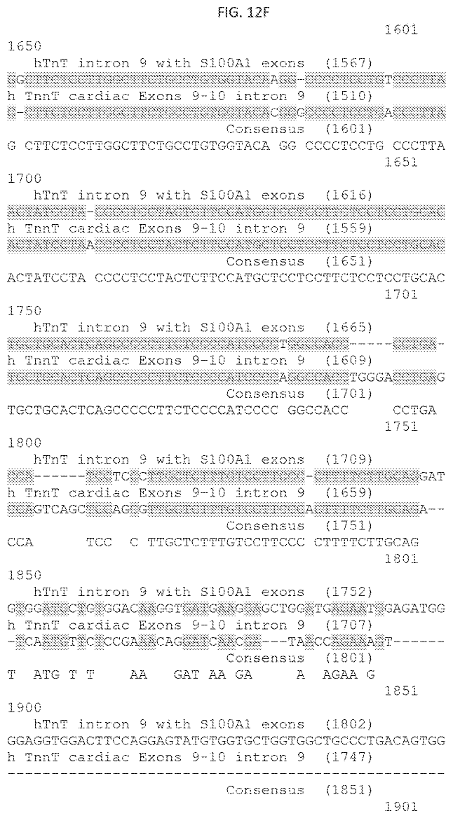

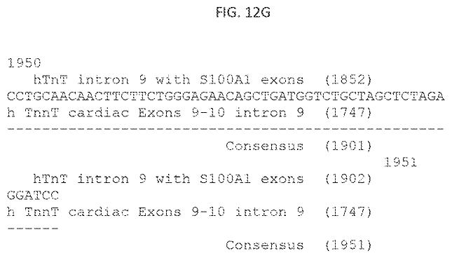

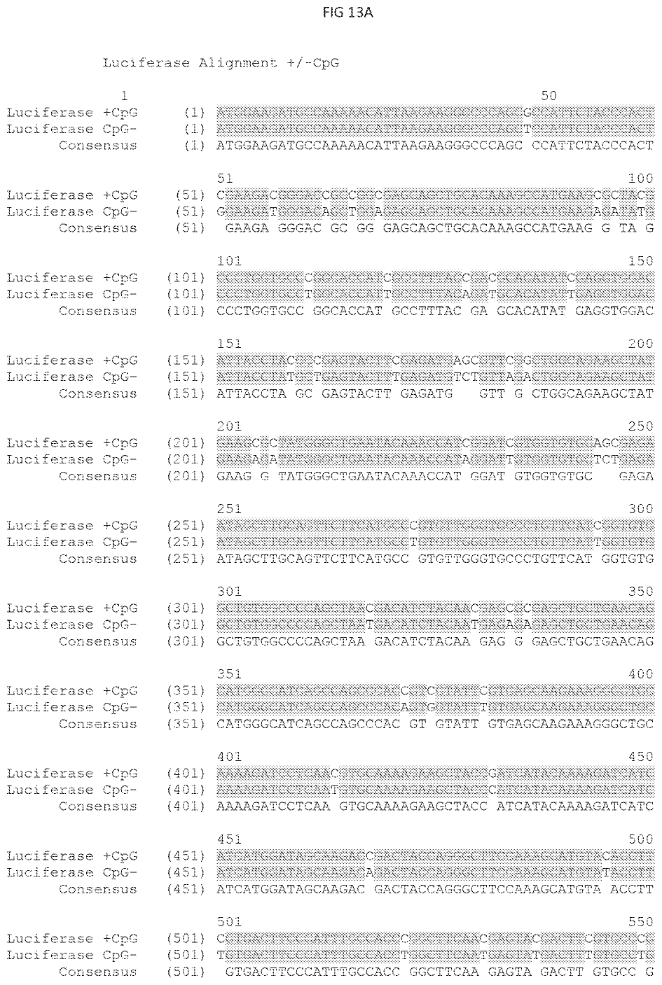

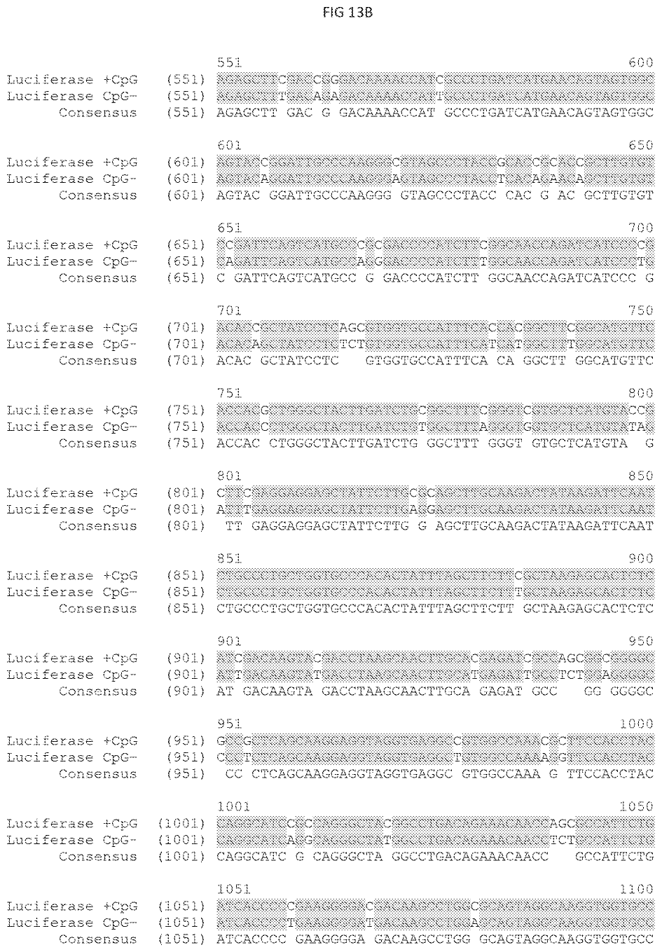

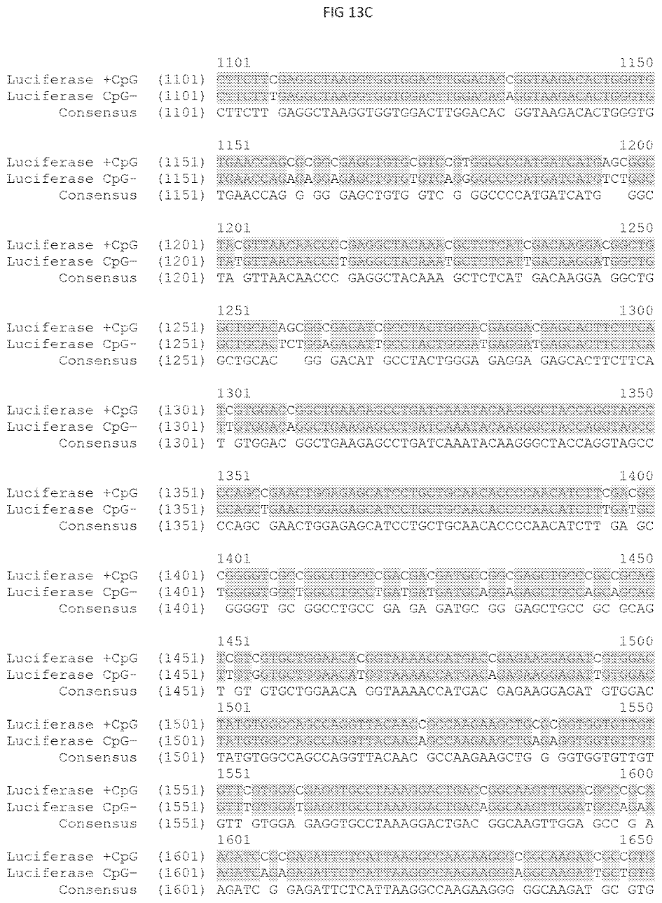

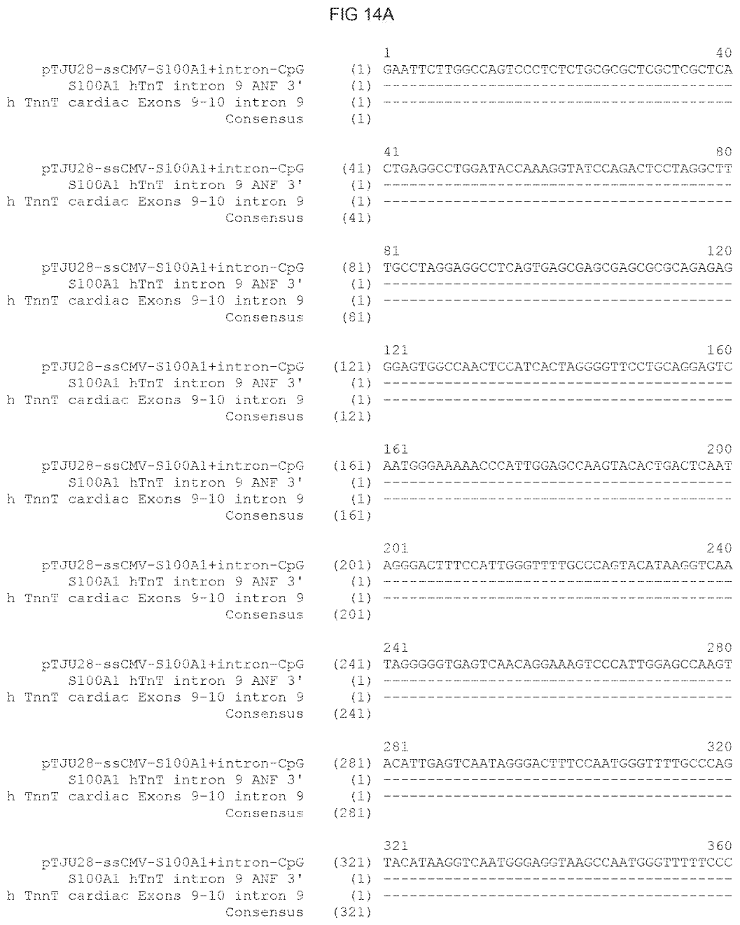

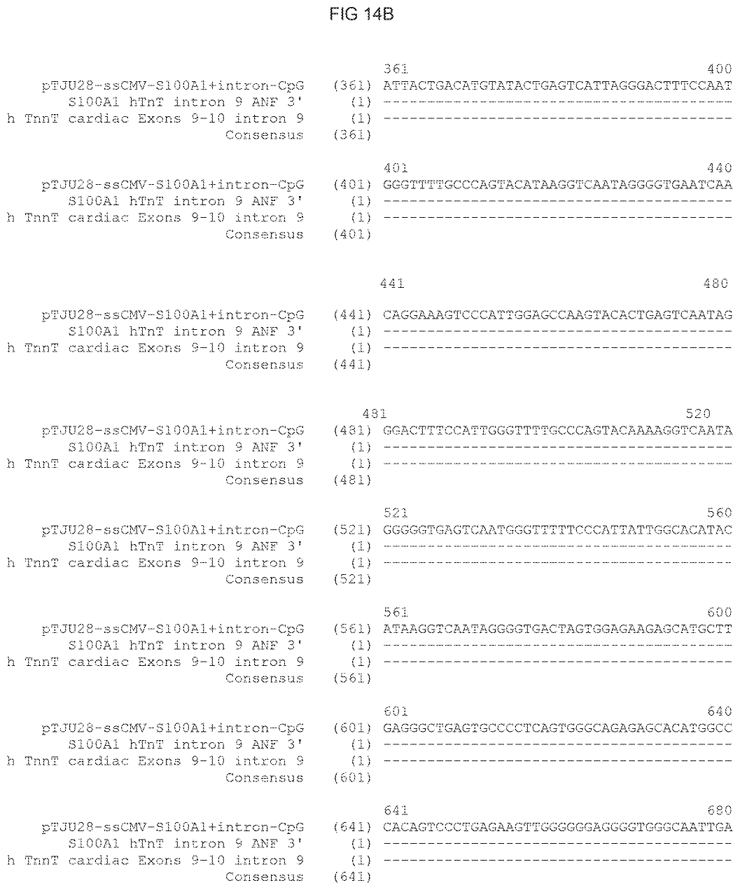

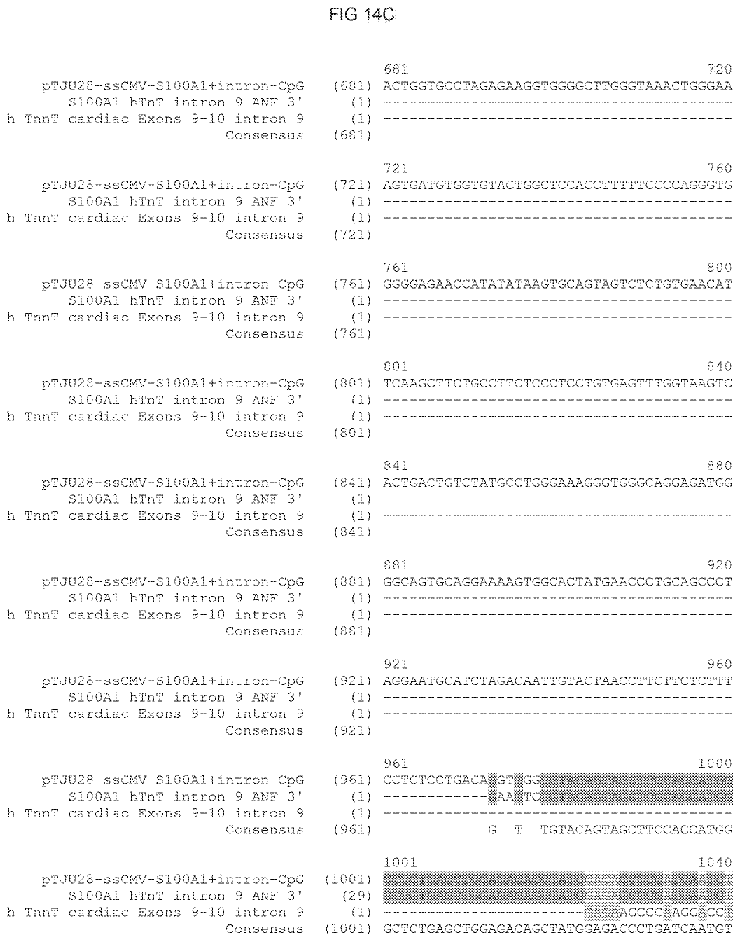

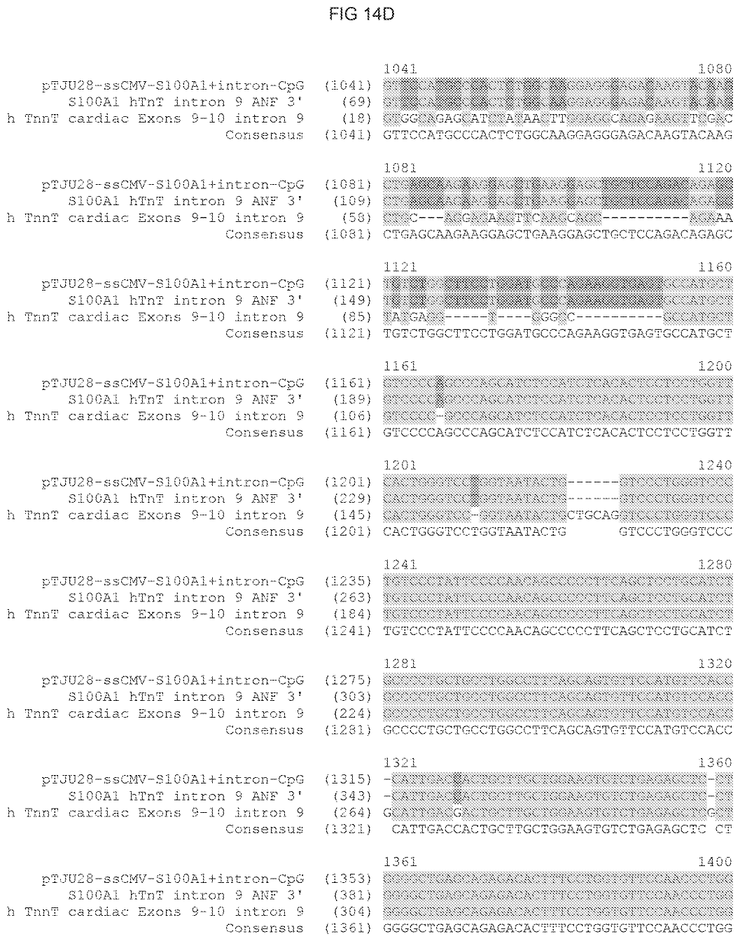

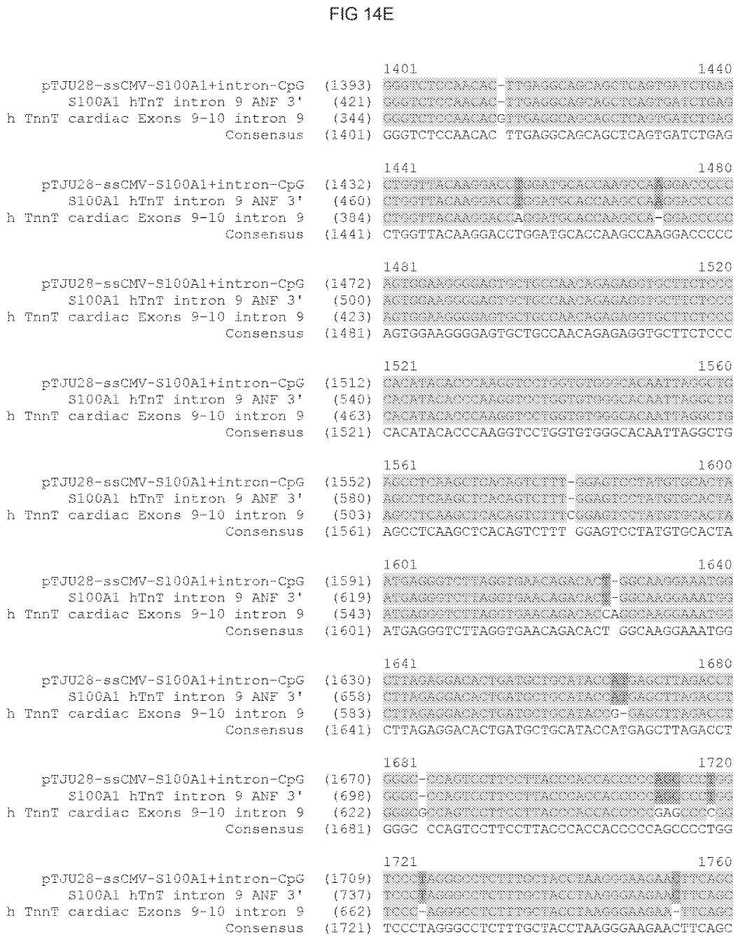

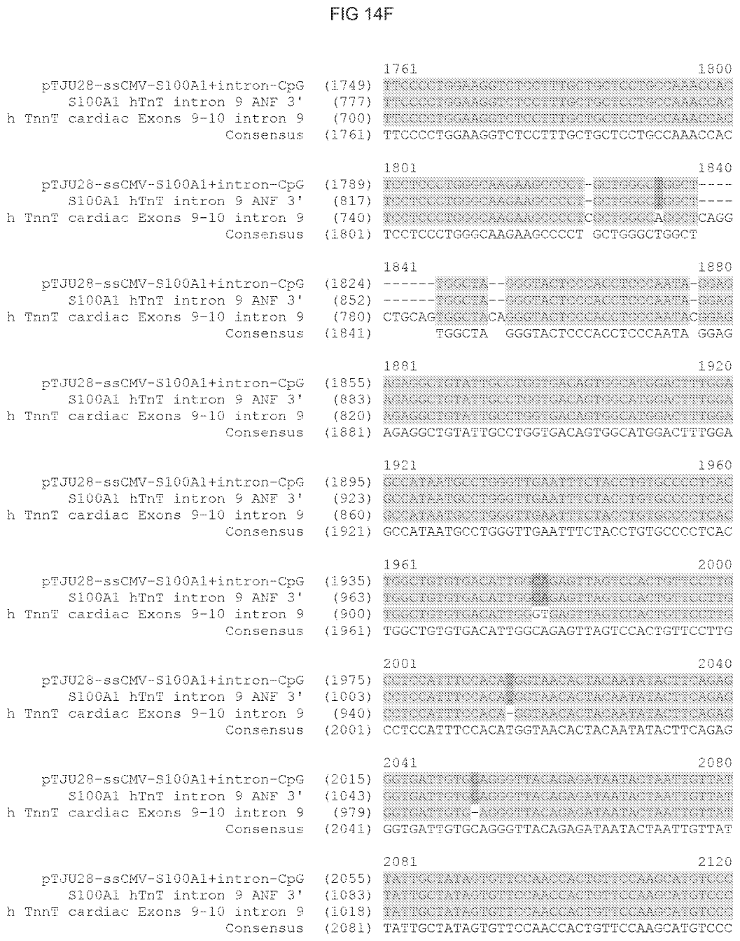

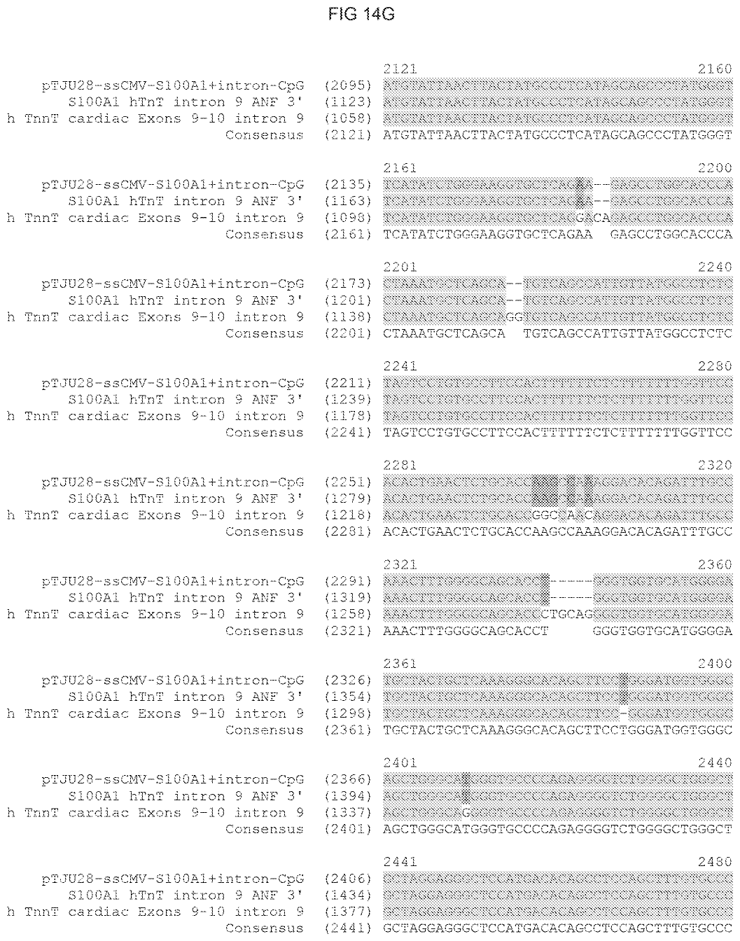

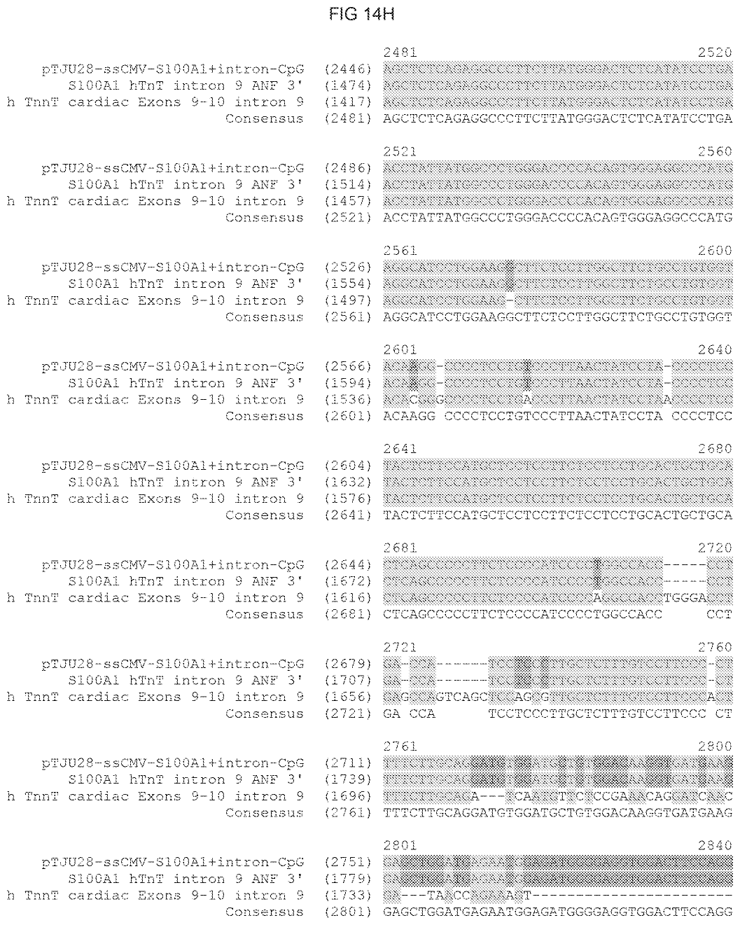

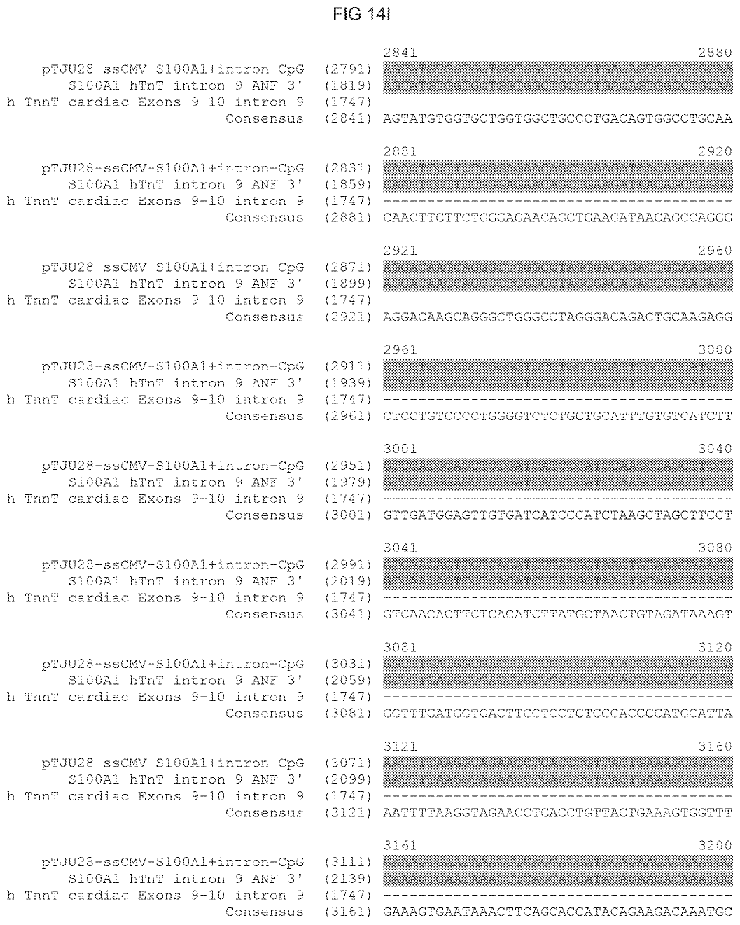

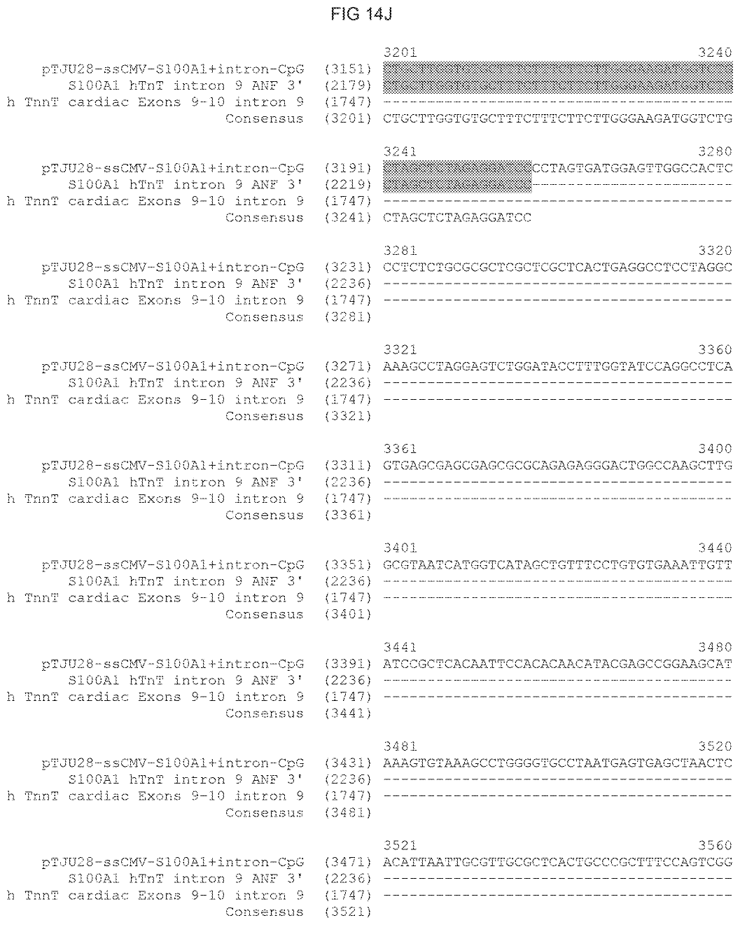

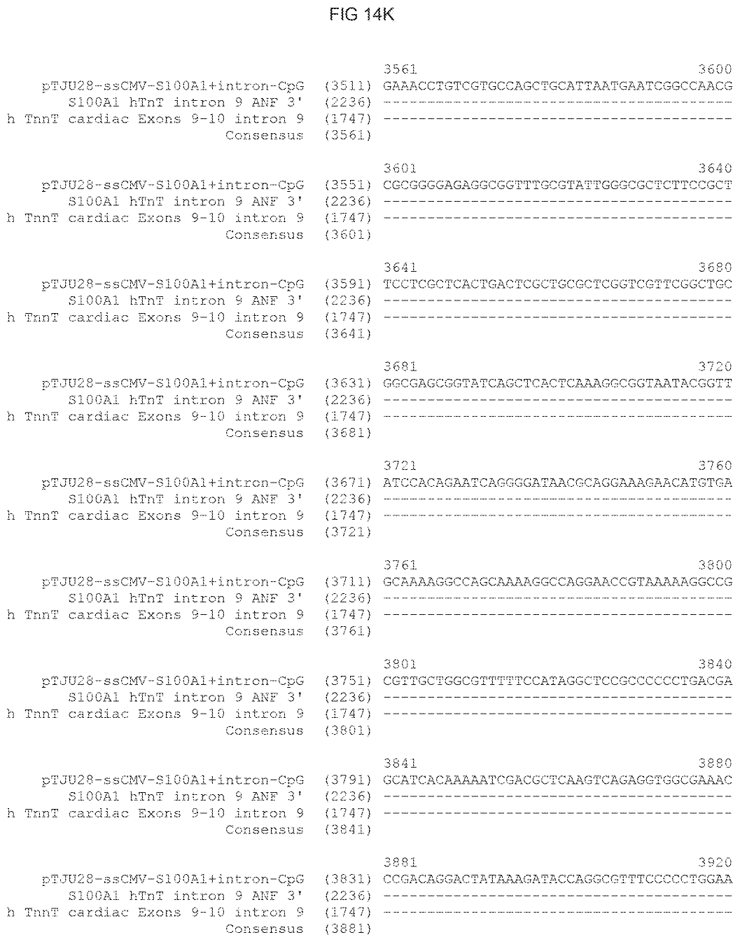

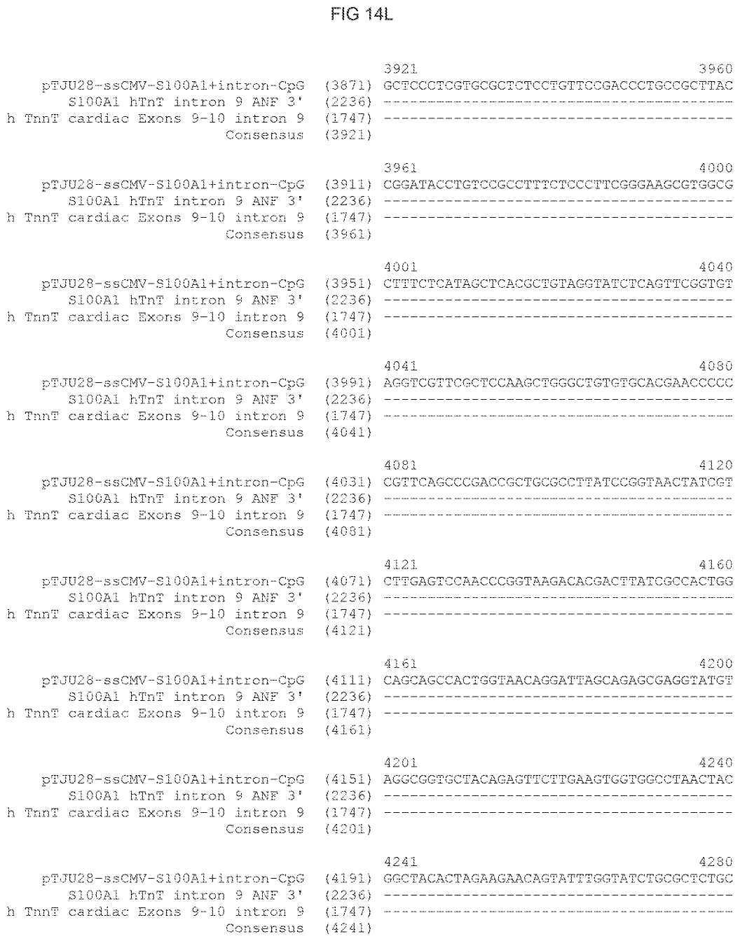

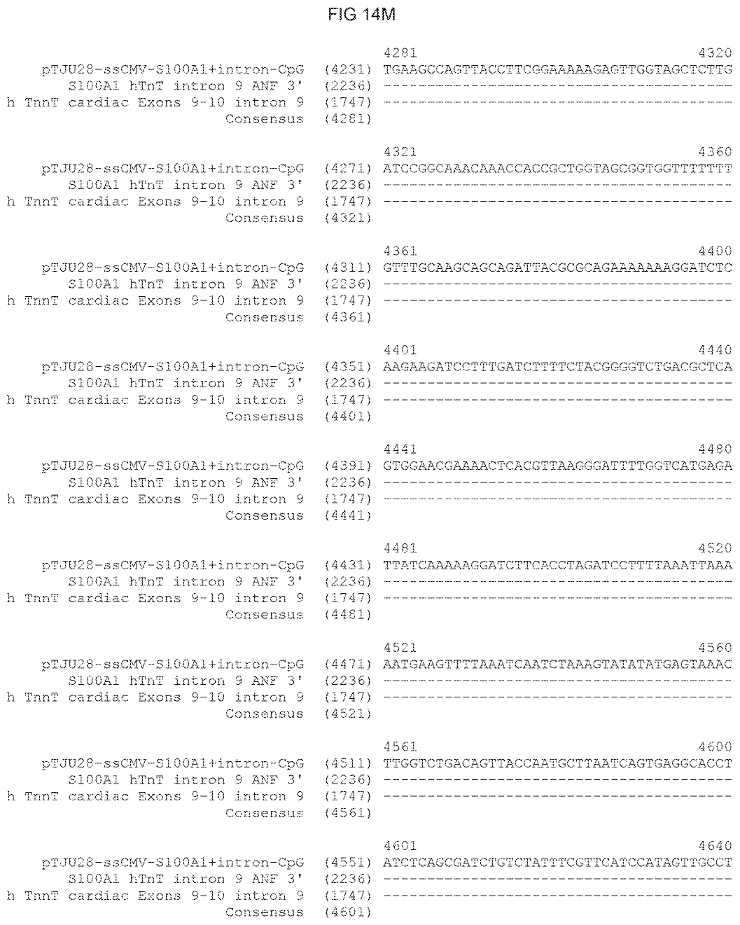

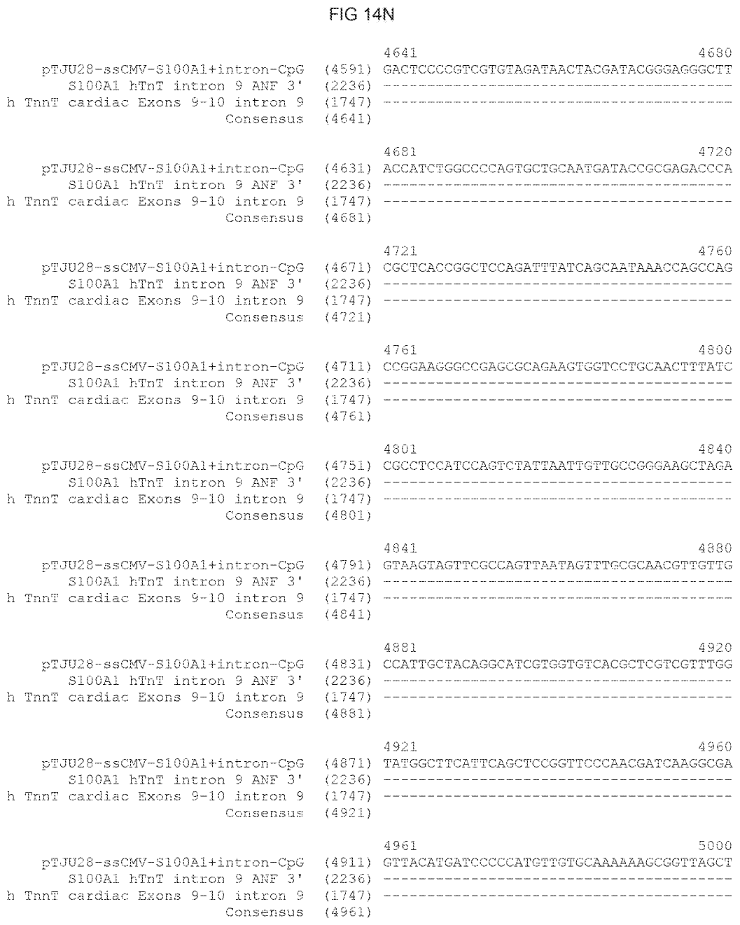

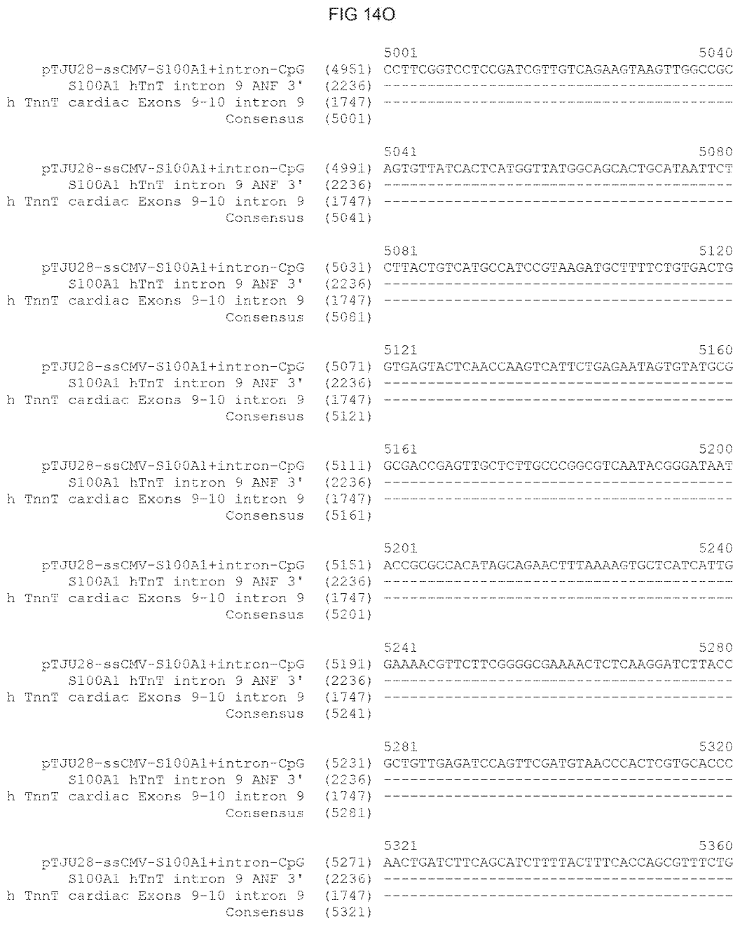

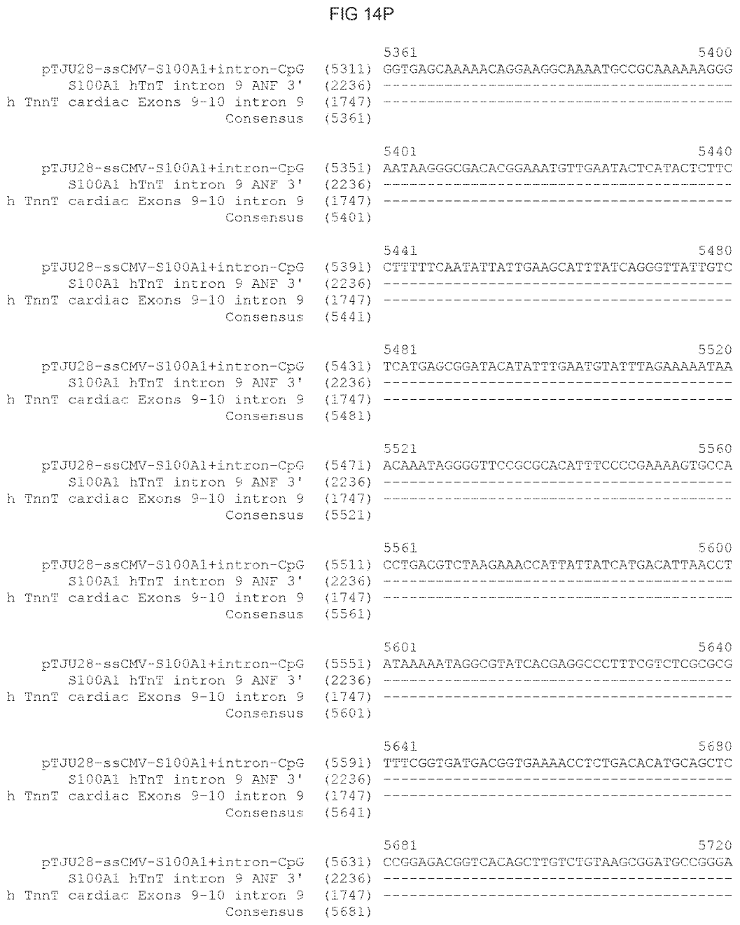

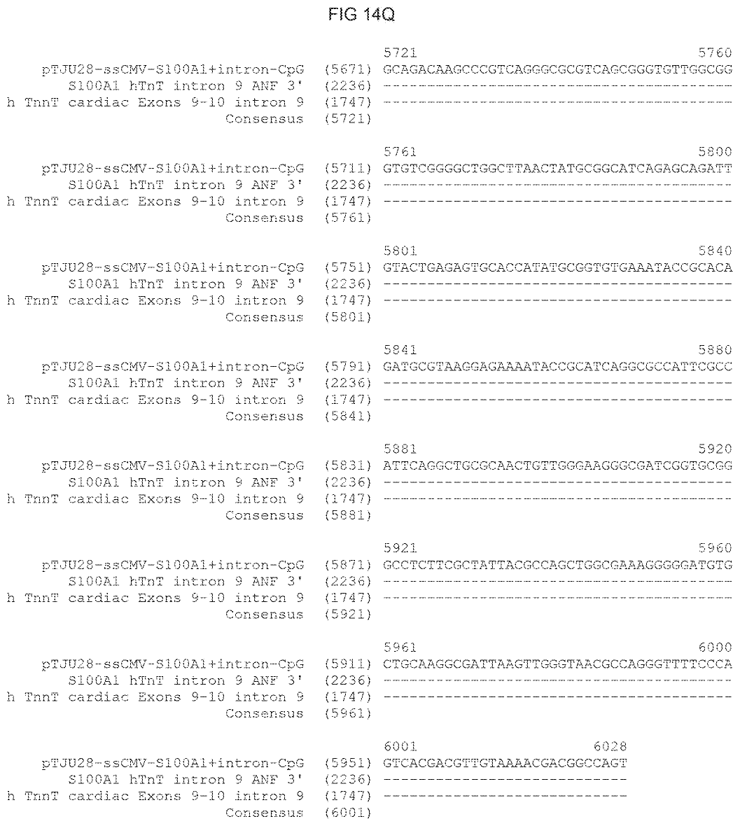

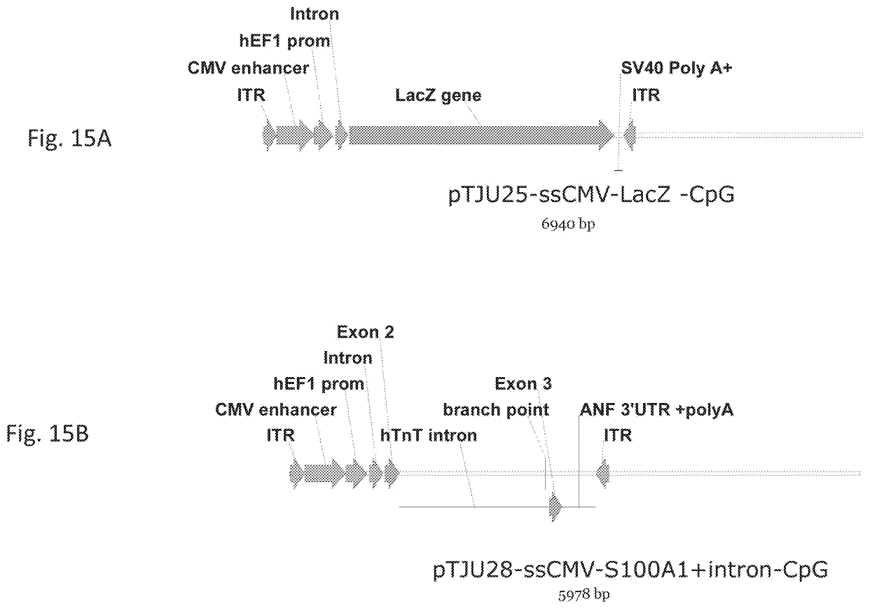

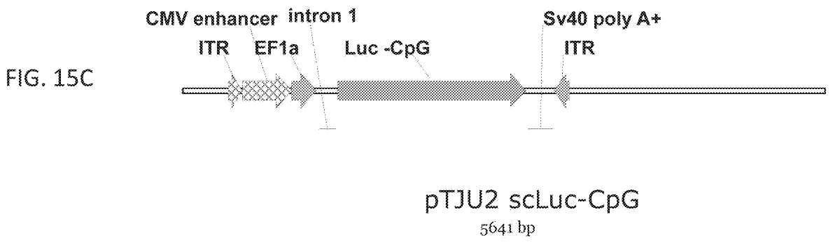

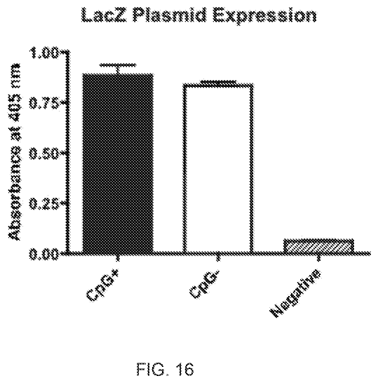

In addition, within the coding region, the interface between triplets must be taken into consideration. For example, if an amino acid triplet ends in a C-nucleotide which is then followed by an amino acid triplet which can start only with a G-nucleotide(e.g., Valine, Glycine, Glutamic Acid, Alanine, Aspartic Acid), then the triplet for the first amino acid triplet is changed to one which does not end in a C-nucleotide. Illustrative CpG− depleted coding sequences are illustrated in FIG. 12 for LacZ and FIG. 13 for luciferase.

Similarly, non-coding sequences carried within the AAV capsid are also altered to be CpG-depleted and/or CpG-free.

Unless otherwise specified, the source of parental terminal repeats, AAV ITRs, and other selected AAV components described herein, may be readily selected from among any AAV, including, without limitation, AAV2, AAV7, AAV9 or another AAV sequences [e.g., US Published Patent Application No. 2011-0263027 A1; US Published Patent Application No. US-2011-0236353A1, U.S. Pat. No. 7,282,199B1; WO 03/042397 A1; WO 2005/033321; WO 2006/110689]. These parental ITRs or other AAV components may be readily isolated from an AAV sequence using techniques available to those of skill in the art of vector genome generation. Such parental AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank®, PubMed®, or the like. These parental AAV sequences are the AAV nucleic acid sequences prior to CpG-depletion via synthetic methods or by site directed mutagenesis.

The wild-type ITRs from known AAV serotypes are CpG rich structures from which

15 CpG dinucleotides are reduced according to the invention without significantly impairing the ability of the ITRs to bind rep protein in a packaging host cell, as required in order to package an expression cassette into an AAV viral vector. For example, the AAV2-5′ and 3′ wild-type ITRs consist of 32 total CpG dinucleotides (16 on each of the 5′ and 3′ ITR). As illustrated herein, these ITRs can each be reduced to 8 CpG dinucleotides and still generate AAV vectors which transduce mouse tissues. However, due to the location of the rep binding sequence on the AAV ITR, it has been found that these AAV2 ITRs may be difficult to modify due to the CpG-free sequence affecting the rep-binding function of the ITRs. Optionally, more CpG-dinucleotides may be reduced on the AAV2-3′ ITR or the AAV2 5′ ITRs, or combinations thereof, which do not affect rep binding and vector packaging. Alternatively, ITRs from other non-AAV2 sources may be selected which differ from AAV2 in the amount of CpG di-nucleotides and the location of these motifs vis-a-vis the rep binding sequence, such as to permit the 5′ AAV ITR, the 3′ AAV ITR, or both, to be rendered CpG-free according to the present invention. Similarly, modified ITRs, e.g., self-complementary ITR, or other sequences (e.g., parvovirus terminal re peats) which are functionally equivalent to AAV 5′ ITRs and/or AAV 3′ ITRs can also be CpG-depleted and used in the present invention.

In addition to the ITRs, other regulatory sequences are desirably CpG-depleted or rendered CpG-free according to the present invention. Such other regulatory sequences include a variety of elements including, e.g., without limitation, untranslated regions, promoter, enhancer, polyA, intron sequences, microRNAs and the like.

For example, the product encoded by the exogenous nucleic acid sequence is typically under the control of a promoter and/or pro moter/enhancer sequence. Desirably, the CpG modifications to the promoters are made in a manner which does not affect the functional characteristics of the promoter and/or enhancer, e.g., without affecting tissue preference.

In an embodiment, where it may not be possible to remove all CpGs from a given nucleic molecule without negatively affecting a desired function, it may be desirable to concentrate on reducing clusters or concentrations of CpG di-nucleotides. For example, prom oter regions have been described as natively containing clusters of CpG di-nucleotides and thus have been used in the development of algorithms which can predict whether removal of such CpG dinucleotides in this region will affect function in an unacceptable manner. Examples of such algorithms include, without limitation, those described in . . . . Y. A. Medvedeva, et al, “Algorithms for CpG Islands Search: New Advantages and Old Problems, in Bioinformatics-Trends and Methodologies”, p. 449-472 (2011); M. Hackenburg, et al, “Prediction of CpG-island function: CpG clustering vs. sliding-wind ow methods”, BMC Genomics, 26 May 2010, 11:327. Synthetic promoter design strategies can be employed to aid in reducing CpG without altering tissue preference.

Similar methods and algorithms can be used to predict which CpG modifications may affect the function of a non-coding region. For other non-coding sequences, however, e.g., an intron, this type of prediction is not required. For example, for an intron, the CpG modifications may be performed so as to ensure that the altered nucleotides do not result in an unwanted coding sequence.

Thus, in one embodiment, an AAV vector of the invention contains within its capsid a nucleic acid sequence which contains a reduced number of CpG di-nucleotides as compared to the sequences for the native elements. In one embodiment, the number of CpG di-nucleotides in the nucleic acid molecule is reduced by at least about 25%, at least about 30%, at least about 45%, at least about 50%, at least about 75%, at least about 80%, at least about 85%, at least about 90%, at least about 95%, or about 95% to 97% CpG-depleted as compared to a nucleic acid molecule having the corresponding native sequences. One or more of the elements of the nucleic acid molecule carried by the AAV vector, e.g., without limitation, the gene coding sequence, a promoter, an enhancer, an intron, the 3′ and 5′ UTRs, may be CpG-free, i.e., lacking any CpG di-nucleotides. In one embodiment, the immune response to the vector is reduced without significant reduction in expression of the gene product. More particularly, in certain instances, changing codons to remove CpG di-nucleotides may be result in less preferential codons being utilized for expression in a given type of host, resulting in lower expression levels of a transgene product. Suitably, codon triplets are selected such that the immune response is reduced while retaining expression levels at a therapeutically or immunologically desired level.

In one embodiment, the CpG-depleted AAV vector retains about 100%, at least about 95%, at least about 85%, at least about 80%, at least about 75% of the protein expression levels as compared to a nucleic acid molecule having the corresponding wild-type sequences, e.g., native ITRs, native exogenous gene sequence and other regulatory sequences. Depending upon the amount of reduction of immune response, a more significant reduction in expression level may be found to be acceptable in order to achieve the therapeutic or immunologic expression levels.

Although to this point the specification has focused on AAV vectors which are CpG-depleted, the approach described herein may be applied to other DNA vectors, and particularly viral vectors which are enveloped or which have capsid proteins. Such viral vectors may include those based on a double-stranded DNA virus, e.g., a virus selected from the group consisting of a baculovirus, poxvirus, herpesvirus or adenovirus, or a single-stranded DNA virus, e.g., another member of the parvovirus family of which AAVis a member.

The term “functional” refers to a product (e.g., a protein or peptide) which performs its native function, although not necessarily at the same level as the native product. The term “functional” may also refer to a gene which encodes a product and from which a desired product can be expressed. A “functional deletion” refers to a deletion which destroys the ability of the product to perform its native function.

Unless otherwise specified (as above), the term fragments includes peptides at least 8 amino acids in length, at least 15 amino acids in length, at least 25 amino acids in length. However, fragments of other desired lengths may be readily utilized depending upon the desired context. Such fragments may be produced recombinantly or by other suitable means, e.g., by chemical synthesis.

The term “percent (%) identity” may be readily determined for amino acid sequences, over the full-length of a protein, or a fragment thereof. Suitably, a fragment is at least about 8 amino acids in length, and may be up to about 700 amino acids. Generally, when referring to “identity”, “homology”, or “similarity” between two different adeno-associated viruses, “identity”, “homology” or “similarity” is determined in reference to “aligned” sequences.

“Aligned” sequences or “alignments” refer to multiple nucleic acid sequences or protein (amino acids) sequences, often containing corrections for missing or additional bases or amino acids as compared to a reference sequence.

When alignments are referenced, or when the CpG-modified sequences are compared to the sequences from the parental sequence prior to modification, conventional alignment techniques may be utilized. There are a number of algorithms known in the art that can be used to measure nucleotide sequence identity, including those contained in the programs described above. As another example, polynucleotide sequences can be compared using Fasta, a program in GCG Version 6.1. Fasta provides alignments and percent sequence identity of the regions of the best overlap between the query and search sequences. For instance, percent sequence identity between nucleic acid sequences can be determined using Fasta with its default parameters (a word size of 6 and the NOPAM factor for the scoring matrix) as provided in GCG Version 6.1, herein incorporated by reference. Similarly, programs are available for performing amino acid alignments. Generally, these programs are used at default settings, although one skilled in the art can alter these settings as needed. Alternatively, one of skill in the art can utilize another algorithm or computer program that provides at least the level of identity or alignment as that provided by the referenced algorithms and programs.

Typically, when an alignment is prepared based upon an amino acid sequence (e.g., an AAV capsid protein), the alignment contains insertions and deletions which are so identified with respect to a reference AAV sequence (e.g., AAV2) and the numbering of the amino acid residues is based upon a reference scale provided for the alignment. However, any given AAV sequence may have fewer amino acid residues than the reference scale. In the present invention, when discus sing the parental sequence, the term “the same position” or the “corresponding position” refers to the amino acid located at the same residue number in each of the sequences, with respect to the reference scale for the aligned sequences. However, when taken out of the alignment, each of the proteins may have these amino acids located at different residue numbers. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used throughout this specification and the claims, the terms “comprise” and “contain” and its variants including, “comprises”, “comprising”, “contains” and “containing”, among other variants, is inclusive of other components, elements, integers, steps and the like . The term “consists of” or “consisting of” are exclusive of other components, elements, integers, steps and the like.

AAV Vectors with Expression Cassettes Having CpG-Mutations

CpG-depleted AAV vectors are described as are methods for generating these sequences. More particularly, the sequence of any of the vector elements described herein for CpG-depletion may be synthesized, generated via site-directed mutagenesis, or in some cases obtained commercially [e.g., CpG-free LacZ may be purchased from Invivogen (wild-type LacZ gene contains about 300 CpG di-nucleotides)). The techniques by which these modifications are made is not a limitation on the present invention. Similarly, once the CpG-depleted sequences are obtained, an AAV vector may be produced using any suitable method.

As previously described, the present invention does not require modifications to the AA V capsid (protein sequence) or to the sequence used to produce same in a packaging host cell. A CpG-depleted AAV vector may be generated according to the invention utilizing any functional AAV capsid. In one embodiment, the capsid is provided by a single AAV source. Suitable AAVs may be selected from among, e.g., AAV2, AAV8, and AAVrh32.22. Still other AAVs may be readily selected from amongst those which have been described [US-2011-0263027A1; US-2011-0236353A1, U.S. Pat. No. 7,282,199 B2; WO 03/042397 A1; WO 2005/033321; WO 2006/110689]. Alternatively, the AAV capsid may be derived from more than one AAV.

Optionally, modified AAV capsid may be utilized to generate a CpG-depleted vector of the invention. For example, an AAV capsid containing a heparin binding site may be modified to ablate the heparin binding motif, e.g., using methods such as those described in WO 2008/027084. Still other modifications to the wild-type AAV capsid may be made, e.g., to enhance production, yield and/or recovery of the capsid [See, e.g., US Published Patent Application 2009-0197338A1 describing “singleton” mutations] or an AAV vector having chimeric or other artificial capsid protein. Other modifications include the modification(s) described in Yew et al, US Published Patent Application No. 2012/0009222, published Jan. 12, 2012.

In one aspect, the invention provides a method of generating a CpG-depleted adeno-associated virus (AAV) having an AAV capsid. Such a method involves culturing a host cell which contains a nucleic acid sequence encoding an AAV capsid; a functional rep gene; and a CpG-depleted expression cassette for packaging into the AAV vector. The components required to be cultured in the host cell to package a CpG-modified expression cassette in an AAV capsid may be provided to the host cell in trans. Alternatively, one or more of the required components (e.g., expression cassette, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contains the rep and/or cap proteins under the control of inducible promoters. Still other stable host cells may be generated by one of skill in the art.

The CpG-depleted expression cassette, rep sequences, cap sequences, and helper functions required for producing the rAAV of the invention may be delivered to the packaging host cell in the form of any genetic element which transfer the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J Virol., 70 :520-532 (1993) and U.S. Pat. No. 5,478,745.

Unless otherwise specified, the source of parental (i.e., wild-type or umodified) AAV ITRs, and other selected AAV components described herein, may be readily selected from among any AAV, including, those identified herein. These ITRs or other AAV components may be readily isolated using techniques available to those of skill in the art from an AAV sequence. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank®, PubMed®, or the like. The AAV ITR and other expression cassette elements are CpG-depleted as described earlier in the specification.

While not repeated in each instance in the following description, it will be readily understood that each of the expression cassette elements may be CpG-depleted according to the present invention and optionally, one or more of these elements (e.g., the coding sequence) may be CpG-free.

### A. The CpG-Depleted Expression Cassette

The CpG-depleted expression cassette is composed of, at a minimum, at least one copy of AAV inverted terminal repeat sequences, a transgene and its regulatory sequences . In one embodiment, both 5′ AAV inverted terminal repeats (ITRs) and 3′ ITRs are included in the expression cassette. In one desirable embodiment, the expression cassette contains ITRs from an AAV sequence other than those which provided AAV capsid sequences. For example, pseudotyped vectors containing both 5′ and 3′ CpG-modified AAV 2 ITRs may be used for convenience. However, a 5′ ITR and/or a 3′ ITR from other suitable AA Vs may be selected and/or CpG-depleted as described herein. In another embodiment, functional equivalents to one or both of the 5′ and 3′ AAV ITRs may be utili zed . Such functional equivalentsfunction as origins of DNA replication and as packaging signals for the viral genome to allow packaging of the DNA molecule into the capsid to form a viral particle. For example, terminal repeats from another parvovirus may provide a functional equivalent and serve as the source of parental termina I repeats (TRs) for CpG-depletion. The CpG-modified expression cassette is packaged into a capsid protein and delivered to a selected host cell.

### 1. The Transgene

The transgene is a nucleic acid sequence, exogenous to the AAV sequences flanking the transgene and the source of the AAV capsid, which encodes a polypeptide, peptide, protein, enzyme, or other product of interest. In one embodiment, the expression cassette carries a nucleic acid sequence, e.g., an RNA. Desirable RNA molecules include tRNA, dsRNA, RNAi, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. This may be useful, e.g., for cancer therapies and vaccines.

The nucleic acid coding sequence is suitably CpG-depleted as described herein and operatively linked to regulatory components in a manner which permits transgene transcription, translation, and/or expression in a host cell.

While any variety of products may be delivered using the constructs of the invention, the invention is particularly well suited for delivery of products useful in diagnosis, treatment, and vaccinati on of conditions associated with conducting airway cells and amelioration of the symptoms thereof.

### 2. Regulatory Elements

In addition to the major elements identified above for the expression cassette, the vector may also include conventional control elements which are CpG-depleted and which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [See, e.g., Boshart et al, Cell, 41:521-530 (1985)], the SV40 promoter, the dihydrofolate reductase promoter, the-actin promoter, the phosphoglycerol kinase (PGK) promoter, and the elongation factor I-alpha (EFI-alpha) promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invivogen, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [International Patent Publication No. WO 98/10088]; the ecdysone insect promoter [No et al, Proc. Natl. Acad. Sci. USA, 93:3346-3351 (1996)), the tetracycline-repressible system [Gossen et al, Proc. Natl. Acad. Sci. USA, 89:5547-5551 (1992)], the tetracycline-inducible system [Gosse n et al, Science, 268:1766-17 69 (1995), See also Harvey et al, Curr. Opin. Chem. Biol., 2:512-518 (1998)), the RU486-inducible system [Wang et al, Nat. Biotech., 15:239-243 (1997) and Wang et al, Gene Ther., 4:432-441 (1997)] and the rapamycin-inducible system [Magari et al, J Clin. Invest., 100:2865-2872(1997)]. Other types of inducible promoters which may be useful in this context are those which are regulated by a specific physiological state, e.g., temperature, acute phase, a particular differentiation state of the cell, or in replicating cells only.

In another embodiment, the native promoter for the transgene will be CpG− depleted and used. This promoter may be preferred when it is desired that expression of the transgene should mimic the native expression, or when expression of the transgene must be regulated temporally or developmentally, in a tissue-specific manner, or in response to specific transcriptional stimuli. In a further embodiment, other CpG-depleted expression control elements, such as enhancer elements, polyadenylation sites or Kozak consensus sequences may also be used to mimic the native expression.

Another embodiment of the transgene includes a CpG-depleted gene operably linked to a tissue-specific promoter. For example, promoters specific for pulmonary tissue and, where available, specific for conducting airway cells, may be used. Examples of such promoters may include, e.g., the forkhead box JI (FOXJI) promoter, polyubiquitin promoter UbC, SAM pointed domain-containing ETS transcription factor (SPDEF) promoter, Clara cell secretory protein/uteroglobin (CCSP/UG) promoter, amongst others. Other lung-specific gene promoters may include, e.g., the surfactant protein B (SPB), surfactant protein C (SPC), and surfactant protein A (SPA), ectogenic human carcinoembryonic antigen (CEA) promoter, Thyroid transcription factor 1 (TTFI) and human surfactant protein Al (hSPAI), amongst others.

Optionally, therapeutically useful transgenes may also include selectable markers or reporter genes that include sequences encoding geneticin, hygromicin or purimycin resistance, among others. Such selectable reporters or marker genes (preferably located outside the viral genome to be rescued by the method of the invention) can be used to signal the presence of the plasmids in bacterial cells, such as ampicillin resistance. Other components of the plasmid may include an origin of replication. Selection of these and other promoters and vector elements are conventional and many such sequences are available.

The combination of the transgene, promoter/enhancer, and AAV ITRs is referred to as an expression cassette for ease of reference herein. Having been provided with the teachings in this specification, the design of a CpG-depleted expression cassette can readily be made by one of skill in the art.

### 3. Delivery of the CpG-Depleted Expression Cassette to a Packaging Host

The CpG-depleted expression cassette can be carried on any suitable vector, e.g., a plasmid, which is delivered to a packaging host cell. The plasmids useful in this invention may be engineered such that they are suitable for replication and, optionally, integration in prokaryotic cells, mammalian cells, or both. These plasmids (or other vectors carrying the 5′ AAV !TR-exogenous molecule-3′ AAV ITR) contain sequences permitting replication of the expression cassette in eukaryotes and/or prokaryotes and selection markers for these systems. Selectable markers or reporter genes may include sequences encoding geneticin, hygromic in or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components, permit high copy episomal replication in the cells. Preferably, the molecule carrying the expression cassette is transfected into the cell, where it may exist transiently. Alternatively, the expression cassette (carrying the 5′ AAV ITR-heterologous molecule-3′ ITR) may be stably integrated into the genome of the host cell, either chromosomally or as an episome. In certain embodiments, the expression cassette may be present in multiple copies, optionally in head-to-head, head-to-tail, or tail-to-tail concatamers. Suitable transfection techniques are known and may readily be utilized to deliver the expression cassette to the host cell.

Generally, when delivering the vector comprising the CpG-depleted expression cassette by transfection, the vector is delivered in an amount from about 5 μg to about 100 μg DNA, about 10 μg to about 50 μg DNA to about 1×10⁴ cells to about 1×10¹³ cells, or about 1×10⁵ cells. However, the relative amounts of vector DNA to host cells may be adjusted by one of ordinary skill in the art, who may take into consideration such factors as the selected vector, the delivery method and the host cells selected.

### B. Rep and Cap Sequences

In addition to the CpG-depleted expression cassette, the host cell contains the sequences which drive expression of an AAV capsid in the host cell and rep sequences of the same source as the source of the AAV ITRs found in the CpG-depleted expression cassette, or a cross-complementing source. The AAV cap and rep sequences may be independently obtained from an AAV source as described above and may be introduced into the host cell in any manner known to one in the art as described above. Additionally, when pseudotyping an AAV vector, the sequences encoding each of the essential rep proteins may be supplied by different AAV sources (e.g., AAV2, AAVS, AAV32/33, AAV5, AAV7, AAV8, AAV9, or one of the other AAV sequences described herein or known in the art). For example, the rep78/6 8 sequences may be from AAV2, whereas the rep52/40 sequences may be from AAV8.

In one embodiment, the host cell stably contains the capsid under the control of a suitable promoter, such as those described above. Most desirably, in this embodiment, the capsid is expressed under the control of an inducible promoter. In another embodiment, the capsid is supplied to the host cell in trans. When delivered to the host cell in trans, the capsid may be delivered via a plasmid that contains the sequences necessary to direct expression of the selected capsid in the host cell. Most desirably, when delivered to the host cell in trans, the plasmid carrying the capsid also carries other sequences required for packaging the rAAV, e.g., the rep sequences.

In another embodiment, the host cell stably contains the rep sequences under the control of a suitable promoter, such as those described above. Most desirably, in this embodiment, the essential rep proteins are expressed under the control of an inducible promoter. In another embodiment, the rep proteins are supplied to the host cell in trans. When delivered to the host cell in trans, the rep proteins may be delivered via a plasmid which contains the sequences necessary to direct expression of the selected rep proteins in the host cell.

Thus, in one embodiment, the rep and cap sequences may be transfected into the host cell on a single nucleic acid molecule and exist stably in the cell as an episome. In another embodiment, the rep and cap sequences are stably integrated into the chromosome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

Optionally, the rep and/or cap sequences may be supplied on a vector that contains other DNA sequences that are to be introduced into the host cells. For instance, the vector may contain the rAAV construct comprising the expression cassette. The vector may comprise one or more of the genes encoding the helper functions, e.g., the adenoviral proteins E1, E2a, and E4 ORF6, and the gene for VAI RNA.

Preferably, the promoter used in this construct may be any of the constitutive, inducible or native promoters known to one of skill in the art or as discuss ed above. In one embodiment, an AAV P5 promoter sequence is employed. The selection of the AA V to provide any of these sequences does not limit the invention.

In another embodiment, the promoter for rep is an inducible promoter, such as are discussed above in connection with the transgene regulatory elements. One preferred promoter for rep expression is the T7 promoter. The vector comprising the rep gene regulated by the T7 promoter and the cap gene, is transfected or transformed into a cell that either constitutively or inducibly expresses the T7 polymerase. See International Patent Publication No. WO 98/10088, published Mar. 12, 1998.

The spacer is an optional element in the design of the vector. The spacer is a DNA sequence interposed between the promoter and the rep gene ATG start site. The spacer may have any desired design; that is, it may be a random sequence of nucleotides, or alternatively, it may encode a gene product, such as a marker gene. The spacer may contain genes which typically incorporate start/stop and polyA sites. The spacer may be a non-coding DNA sequence from a prokaryote or eukaryote, a repetitive non-coding sequence, a coding sequence without transcriptional controls or a coding sequence with transcriptional controls. Two exemplary sources of spacer sequences are the “A, phage ladder sequences or yeast ladder sequences, which are available commercially, e.g., from Gibco or Invitrogen, among others. The spacer may be of any size sufficient to reduce expression of the rep78 and rep68 gene products, leaving the rep52, rep40 and cap gene products expressed at normal levels. The length of the spacer may therefore range from about 10 bp to about 10.0 kbp, preferably in the range of about 100 bp to about 8.0 kbp. To reduce the possibility of recombination, the spacer is preferably less than 2 kbp in length; however, the invention is not so limited.

Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell. The methods employe d for constructing embodiments of this invention are convention al genetic engineering or recombinant engineering techniques such as those described in the references above. While this specification provides illustrative examples of specific constructs, using the information provided herein, one of skill in the art may select and design other suitable constructs, using a choice of spacers, PS promoters, and other elements, including at least one translational start and stop signal, and the optional addition of polyadenylation sites.

In another embodiment of this invention, the rep or cap protein may be provided stably by a host cell.

### C. The Helper Functions

The packaging host cell also requires helper functions in order to package the rAAV of the invention. Optionally, these functions may be supplied by a herpesvirus. Most desirably, the necessary helper functions are each provided from a human or non-human primate adenovirus source, such as those described above and/or are available from a variety of sources, including the American Type Culture Collection (ATCC), Manassas, Va. (US). In one currently preferred embodiment, the host cell is provided with and/or contains an E1 a gene product, an E1b gene product, an E2a gene product, and/or an E4 ORF6 gene product. The host cell may contain other adenoviral genes such as VAI RNA, but these genes are not required. In a preferred embodiment, no other adenovirus genes or gene functions are present in the host cell.

The adenovirus E1a, E1b, E2a, and/or E4ORF6 gene products, as well as any other desired helper functions, can be provided using any means that allows their expression in a cell. Each of the sequences encoding these products may be on a separate vector, or one or more genes may be on the same vector. The vector may be any vector known in the art or disclosed above, including plasmids, cosmids and viruses. Introduction into the host cell of the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by other means, e.g., by exogenously added factors.

### D. Host Cells and Packaging Cell Lines

The host cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The requirements for the cell used is that it not carry any adenovirus gene other than E1, E2a and/or E4 ORF6; it not contain any other virus gene which could result in homologous recombination of a contaminating virus during the production of rAAV; and it is capable of infection or transfection of DNA and expression of the transfected DNA. In a preferred embodiment, the host cell is one that has rep and cap stably transfected in the cell.

One host cell useful in the present invention is a host cell stably transformed with the sequences encoding rep and cap, and which is transfected with the adenovirus E1, E2a, an E4ORF6 DNA and a construct carrying the expression cassette as described above. Stable rep and/or cap expressing cell lines, such as B-50 (International Patent Application Publication No. WO 99/15685), or those described in U.S. Pat. No. 5,658,785, may also be similarly employed. Another desirable host cell contains the minimum adenoviral DNA which is sufficient to express E4 ORF6.

The preparation of a host cell according to this invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods which provide the desired nucleotide sequence.

Introduction of the molecules (as plasmids or viruses) into the host cell may also be accomplished using techniques known to the skilled artisan and as discussed throughout the specification. In preferred embodiment, standard transfection techniques are used, e.g., CaPO₄ transfection or electroporation, and/or infection by hybrid adenovirus/ AAV vectors into cell lines such as the human embryonic kidney cell line HEK 293 (a human kidney cell line containing functional adenovirus E I genes which provides trans-acting El proteins).

Once generated, the CpG-depleted AAV vectors may be isolated and purified using techniqu es known to those of skill in the art and used to prepare suitable formulation.

### Pharmaceutical Compositions and Uses Therefor

These CpG-depleted AAV vectors may be prepared into a pharmaceutical composition, typically in the form of a suspension with a pharmaceutically acceptable carrier. The CpG-depleted AAV may be delivered to host cells according to published methods. The AAV may be administered to a human or non-human mammalian patient. Suitable carriers may be readily selected by one of skill in the art in view of the indication for which the transfer virus is directed.

For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water.

Optionally, the compositions may contain, in addition to the AAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The vectors or AAV targeting moieties are administered in sufficient amounts to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the lung, liver, skeletal muscle, eye, heart), oral, inhalation, intranasal, intratracheal, intraarterial, intraocular, intravenous, intramuscular, subcutaneous, intradermal, and other parental routes of administration. Routes of administration may be combined, if desired.

Dosages of the viral vector or targeting moiety will depend primarily on factors such as the condition being treated, the age, weight and health of the patient, and may thus vary among patients. For example, a therapeutically effective human dosage of the viral vector is generally in the range of from about 0.1 mL to about 100 mL of solution containing concentrations of from about 1×10⁹ to 1×10¹⁶ genomes virus vector. A preferred human dosage for delivery to large organs (e.g., liver, muscle, heart and lung) may be about 5×10¹⁰ to 5×10¹³ AAV genomes, at a volume of about 1 to 100 mL. A preferred dosage for delivery to eye is about 5×10⁹ to 5×10¹² genome copies, at a volume of about 0.1 mL to 1 mL. For example, a therapeutically effective human dosage of an airway conducting cell targeting moiety is generally in the range of about 100 μg to about 10 mg of the moiety . This may be delivered in solution, e.g., in about 0.1 mL to about 100 mL of solution. Optionally, dosage regimens similar to those described for therapeutic purposes may be utilized for immunization using the compositions of the invention. For example, an immunogically effective human dosage of the viral vector is generally in the range of from about 0.1 mL to about 100 mL of solution containing concentrations of from about 1×10⁹ to 1×10¹⁶ genomes virus vector. One human dosage for delivery to large organs (e.g., liver, muscle, heart and lung) may be about 5×10¹⁰ to 5×10¹³ AAV genomes, at a volume of about 1 to 100 mL. One dosage for delivery to eye is about 5×10⁹ to 5×10¹² genome copies, at a volume of about 0.1 mL to 1 mL.

In another embodiment, an amount of CpG-depleted AAV composition is administered at an effective dose that is in the range of about 1×10⁸ genome copies (GC) CpG-depleted AAV/kilogram (kg) to about 1×10¹⁴ GC/kg, and preferably 1×10¹¹ GC CpG-depleted AAV/kg to 1×10¹³ GC CpG-depleted AAV/kg to a human patient. Preferably, the amount of CpG-depleted virus composition administered is 1×10⁸ GC/kg, 5×10⁸ GC/kg, 1×10⁹ GC/kg, 5×10⁹ GC/kg, 1×10¹⁰ GC/kg, 5×10¹⁰ GC/koo, 1×10¹¹ GC/kg, 5×10¹¹ GC/kg, or 1×10¹² GC/kg, 5×10¹² GC/kg, 1×10¹³ GC/kg, 5×10¹³ GC/kg, 1×10¹⁴ GC/kg.

These doses can be given once or repeatedly, such as daily, every other day, weekly, biweekly, or monthly, or until adequate transgene expression is detected in the patient. In an embodiment, virus compositions are given once weekly for a period of about 4-6 weeks, and the mode or site of administration is preferably varied with each administration. Repeated injection is most likely required for complete ablation of transgene expression. The same site may be repeated after a gap of one or more injections. Also, split injections may be given. Thus, for example, half the dose may be given in one site and the other half at another site on the same day.

Examples of therapeutic products and immunogenic products for delivery by include those previously described for delivery via AAV constructs including those in, e.g., WO 2011/126808A2 and US Published Patent Application No. 2009/0227030-AI, incorporated by reference herein. These CpG-depleted AAV vector may be used for a variety of therapeutic or vaccinal regimens, as described herein. Additionally, these CpG-depleted vectors may be delivered in combination with one or more other vectors or active ingredients in a desired therapeutic and/or vaccinal regimen. For example, the CpG-depleted AAV compositions of the invention can be administered to a human or non-human subject by any method described in the following patents and patent applications that relate to methods of using AAV vectors in various therapeutic applications: U.S. Pat. Nos. 7,282,199; and 7,198,951; U.S. Patent Application Publication Nos. US 2008-0075737; US 2008-0075740; International Patent Application Publication Nos. WO 2003/024502; WO 2004/108922; WO 20051033321, each of which is incorporated by reference in its entirety.

### Treatment of Diseases and Disorders and Therapeutic Regimens

The invention provides methods for treating any disease or disorder that is amenable to gene therapy. In one embodiment, “treatment” or “treating” refers to an amelioration of a disease or disorder, or at least one discernible symptom thereof. In another embodiment, “treatment” or “treating” refers to an amelioration of at least one measurable physical parameter associated with a disease or disorder, not necessarily discernible by the subject. In yet another embodiment, “treatment” or “treating” refers to inhibiting the progression of a disease or disorder, either physically, e.g., stabilization of a discernible symptom, physiologically, e.g., stabilization of a physical parameter, or both. Other conditions, including cancer, immune disorders, and veterinary conditions, may also be treated.

Types of diseases and disorders that can be treated by methods of the present invention include, but are not limited to age-related macular degeneration; diabetic retinopathy; infectious diseases e.g., HIV, pandemic flu, category 1 and 2 agents of biowarfare, or any new emerging viral infection; autoimmune diseases; cancer; multiple myeloma; diabetes; systemic lupus erythematosus (SLE); hepatitis C; multiple sclerosis; Alzheimer's disease; Parkinson's disease; amyotrophic lateral sclerosis (ALS), Huntington's disease; epilepsy; chronic obstructive pulmonary disease (COPD); joint inflammation, arthritis; myocardial infarction (MI); congestive heart failure (CHF); hemophilia A; or hemophilia B.

Infectious diseases that can be treated or prevented by the methods of the present invention are caused by infectious agents including, but not limited to, viruses, bacteria, fungi, protozoa, helminths, and parasites. The invention is not limited to treating or preventing infectious diseases caused by intracellular pathogens. Many medically relevant microorganisms have been described extensively in the literature, e.g., See C. G. A Thomas, Medical Microbiology, Bailliere Tindall, Great Britain 1983, the entire contents of which are hereby incorporated herein by reference.

### Therapeutic Transgenes

Useful therapeutic products encoded by the transgene include hormones and growth and differentiation factors including, without lim itation, ins ulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-1 and IGF-II), any one of the transforming growth factor a. superfamily, including TGFα, activins, inhibins, or any of the bone morphogenic proteins (BMP) BMPs 1-15 as well as TGFβ proteins, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors a and p, interferons α, β, TGFβ and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecule s, as well as engineered immunoglobulins and J\1HC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein(VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcript ion factors such as jun, fos, max, mad, serum responsefactor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP I, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-I antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding P-glucuronidase (GUSB)).

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. No. 6,200,560 and U.S. Pat. No. 6,221,349). The Factor VIII gene codes for 2351 amino acids and the protein has six domains, designated from the amino to the terminal carboxy terminus as Al-A2-B-A3-Cl-C2 (Wood et al, Nature, 312:330 (1984); Vehar et al., Nature 312:337 (1984); and Toole et al, Nature, 342:337 (1984)]. Human Factor VIII is processed within the cell to yield a heterodimer primaril y comprising a heavy chain containing the Al, A2 and B domains and a light chain containing the A3, C1 and C2 domains. Both the single chain polypeptide and the heterodimer circulate in the plasma as inactive precursors, until activated by thrombin cleavage between the A2 and B domains, which releases the B domain and results in a heavy chain consisting of the Al and A2 domains. The B domain is deleted in the activated procoagulant form of the protein. Additionally, in the native protein, two polypeptide chains (“a” and “b”), flanking the B domain, are bound to a divalent calcium cation.

In some embodiments, the minigene comprises first 57 base pairs of the Factor VIII heavy chain that encodes the 10 amino acid signal sequence, as well as the human growth hormone (hGH) polyadenylation sequence. In alternative embodiments, the minigene further comprises the Al and A2 domains, as well as 5 amino acids from the N-terminus of the B domain, and/or 85 amino acids of the C-terminus of the B domain, as well as the A3, Cl and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

As used herein, a therapeutically effective amount is an amount of AAV vector that produces sufficient amounts of Factor VIII to decrease the time it takes for a subject's blood to clot. Generally, severe hemophiliacs having less than 1% of normal levels of Factor VIII have a whole blood clotting time of greater than 60 minutes as compared to approximately 10 minutes for non-hemophiliacs.

The present invention is not limited to any specific Factor VIII sequence. Many natural and recombinant forms of Factor VIII have been isolated and generated. Examples of naturally occurring and recombinant forms of Factor VII can be found in the patent and scientific literature including, U.S. Pat. Nos. 5,563,045, 5,451,521, 5,422,260, 5,004,803, 4,757,006, 5,661,008, 5,789,203, 5,681,746, 5,595,886, 5,045,455, 5,668,108, 5,633,150, 5,693,499, 5,587,310, 5,171,844, 5,149,637, 5,112,950, and 4,886,876, WO 94/11503, WO 87/07144, WO 92/16557, WO 91/09122, WO 97/03195, WO 96/21035, WO 91/07490, EP 0 672 138, EP 0 270 618, EP 0 182 448, EP 0 162 067, EP 0 786 474, EP 0 533 862, EP 0 506 757, EP 0 874 057, EP 0 795 021, EP 0 670 332, EP 0 500 734, EP 0 232 112, EP 10 0 160 457, Sanberg et al., XX:th Int. Congress of the World Fed. Of Hemophilia (1992), and Lind et al., Eur. J. Biochem., 232:19 (1995).

Nucleic acids sequences coding for the above-described Factor VIII can be obtained using recombinant methods or by deriving the sequence from a vector known to include the same. Furthermore, the desired sequence can be isolated directly from cells and tissues containing the same, using standard techniques, such as phenol extraction and PCR of cDNA or genomic DNA [See, e.g., Sambrook et al]. Nucleotide sequences can also be produced synthetically, rather than cloned. The complete sequence can be assembled from overlapping oligonucleotides prepared by standard methods and assembled into a complete coding sequence [See, e.g., Edge, Nature 292:757 (1981); Nambari et al, Science, 223:1299 (1984); and Jay et al, J. Biol. Chem. 259:6311 (1984).

Factor VIII from humans and non-human animals, including but not limited to companion animals (e.g., canine, felines, and equines), livestock (e.g., bovines, caprines and ovines), laboratory animals, marine mammals, large cats, etc. are encompassed. The AAV vectors may contain a nucleic acid coding for fragments of Factor VIII which is itself not biologically active, yet when administered into the subject improves or restores the blood clotting time. For example, as discussed above, the Factor VIII protein comprises two polypeptide chains: a heavy chain and a light chain separated by a B-domain which is cleaved during processing. As demonstrated by the present invention, co-tranducing recipient cells with the Factor VIII heavy and light chains leads to the expression of biologically active Factor VIII. Because, however, most hemophiliac s contain a mutation or deletion in only one of the chain (e.g., heavy or light chain), it may be possible to administer only the chain defective in the patient to supply the other chain.

Other useful gene products include non-naturally occurring polypeptides, such as chimeric or hybrid polypeptideshaving a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor associated polypeptides can be used as target polypeptides such as polypeptides which are found at higher levels in tumor cells including the polypeptide recognized by monoclonal antibody 17 I A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, derrnatomyositis, psoriasis, vasculitis, Wegener's granulomatosis, Crohn's disease and ulcerative colitis. Each of these diseases is characterized by T cell receptors (TCRs) and antibodies (Ab) that bind to endogenous antigens and initiate the inflammatory cascade associated with autoimmune diseases.

### Immunogenic Transgenes

Suitably, the AAV vectors of the inventio n avoid the generation of immune responses to the AAV sequences contained within the vector. However, these vectors may nonetheless be formulated in a manner which permits the expression of a transgene carried by the vectors to induce an immune response to a selected antigen . For example, in order to promote an immune response, the transgene may be expressed from a constitutive promoter, the vector can be adjuvanted as described herein, the transgene can optionally be modified to express more CpGs to induce a greater immune response, and/or the vector can be put into degenerating tissue.

Examples of suitable immunogenic transgenes include those selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Other viral families include the astroviruses and the calcivirus family. The calcivirus family encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cats), feline enteric coronavirus (cat), canine coronavirus (dog), and human respiratory coronaviruses, which may cause the common cold and/or non A, B or C hepatitis, and which include the putative cause of sudden acute respiratory syndrome (SARS). Within the coronavirus family, target antigens include the El (also called Mor matrix protein), E2 (also called S or Spike protein), E3 (also called HE or hemagglutin elterose) glycoprotein (not present in all coronaviruses), or N (nucleocapsid). Still other antigens may be targeted against the arterivirus family and the rhabdovirus family. The rhabdovirus family includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the NI protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. Another source of antigens is the bornavirus family. The reovirus family includes the genera reovirus, rotavirus (which causes acute gastroenteritis in children), orbiviruses, and cultivirus (Colorado Tick fever, Lebombo (humans), equine encephalosis, blue tongue). The retrovirus family includes the sub family oncorivirinal which encompasses such human and veterinary diseases as feline leukemia virus, HTLVI and HTLVII, lentivirinal (which includes HIV, simian immunodeficiency virus, feline immunodeficiency virus, equine infectious anemia virus, and spumavirinal). The papovavirus family includes the sub-family polyomaviruses (BKU and JCU viruses) and the sub family papillomavirus (associated with cancers or malignant progression of papilloma). The adenovirus family includes viruses (EX, AD7, ARD, O. B.) which cause respiratory disease and/or enteritis. The parvovirus family feline parvovirus (feline enteritis), feline panleucopeniavirus, canine parvovirus, and porcine parvovirus. The herpesvirus family includes the sub family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub family gamma herpesvirinae, which includes the genera lymphocryptovirus, EBV (Burkitts lymphoma), human herpesviruses 6A, 6B and 7, Kaposi's sarcoma-associated herpesvirus and cercopithecine herpesvirus (B virus), infectious rhinotracheitis, Marek's disease virus, and rhadinovirus. The poxvirus family includes the sub family chordopoxvirinae, which encompasses the genera orthopoxvirus (Variola major (Smallpox) and Vaccinia (Cowpox)), parapoxvirus, avipoxvirus, capripoxvirus, leporipoxvirus, suipoxvirus, and the sub family entomopoxvirinae. The hepadnavirus family includes the Hepatitis B virus. One unclassified virus which may be suitable source of antigens is the Hepatitis delta virus, Hepatitis E virus, and prions. Another virus which is a source of antigens is Nipan Virus. Still other viral sources may include avian infectious bursa! disease virus and porcine respiratory and reproductive syndrome virus. The alphavirus family includes equine arteritis virus and various Encephalitis viruses.

The present invention may also encompass immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram positive cocci include pneumococci; staphylococci (and the toxins produced thereby, e.g., enterotoxin B); and streptococci. Pathogenic gram negative cocci include meningococcus; gonococcus. Pathogenic enteric gram negative bacilli include enterobacteriaceae; pseudomonas, acinetobacteria and eikenella; melioidosis ; salmonella; shigella; haemophilus; moraxella; H. ducreyi (which causes chancroid); brucella species (brucellosis); Francisella tularensis (which causes tularemia); Yersinia pestis (plague) and other yersinia (pasteurella); streptobacillus moniliformis and spirillum; Gram-positive bacilli include listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism (Clostridum botulinum and its toxin); Clostridium perfringens and its epsilon toxin; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include glanders (Burkholderia mallei); actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infect ions include Typhus fever, Rocky Mountain spotted fever, Q fever (Coxiella burnetti), and Rickettsialpox. Examples of mycoplasma and chlamydia) infections include: mycoplasma pneumoniae; lymphogranuloma venereum; psittacosis; and perinatal chlamydia! infections. Pathogenic eukaryotes encompass pathogenic protozoans and helminths and infections produced thereby include : amebiasis; malaria; leishmaniasis; trypanosomiasis; toxoplasmosis; Pneumocystis carinii; Trichans; Toxoplasma gondii; babesiosis; 5

giardiasis; trichinosis; filariasis; schistosomiasis; nematodes; trematodes or flukes; and cestode (tapeworm) infections.

Many of these organisms and/or the toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), variola major (small pox), Francisella tularensis (tularemia), and viral hemorrhagic fevers [filoviruses (e.g., Ebola, Marburg], and arenaviruses [e.g., Lassa, Mac hupo]), all of which are currently classified as Category A agents; Coxiella burnetti (Q fever); Brucella species (brucellosis), Burkholderia mallei (glanders), Burkholderia pseudomallei (meloidosis), Ricinus communis and its toxin (ricin toxin), Clostridium perfringens and its toxin (epsilon toxin), Staphylococcus species and their toxins (enterotoxin B), Chlamydia psittaci (psittacosis), water safety threats (e.g., Vibrio cholerae, Crytosporidium parvum), Typhus fever (Richettsia powazekii), and viral encephalitis (alphaviruses, e.g., Venezuelan equine encephalitis; eastern equine encephalitis; western equine encephalitis); all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vecto rs and other constructs described herein are useful to deliver antigens from these organisms, viruses, their toxins or other by-products, which will prevent and/or treat infection or other adverse reactions with these biological agents.

The vectors of the invention can be used to deliver immunogens . In rheumatoid arthritis (RA), several specific variable regions of T-cell receptors (TCRs) which are involved in the disease have been characterized. These TCRs include V 3, V 14, V 17 and V 17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V 7 and V 10. Thus, delivery of a nucleic acid sequence that encodes at least one of these polypeptides will elicit an immune response that will target T cells involved in MS. In scleroder ma, several specific variable regions of TCRs which are involved in the disease have been characterized. These TCRs include V 6, V 8, V 14 and V 16, V 3C, V 7, V 14, V 15, V 16, V 28 and V 12. Thus, delivery of a nucleic acid molecule that encodes at least one of these polypeptides will elicit an immune response that will target T cells involved in scleroderma.

A CpG-modified AAV viral vector of the invention provides an efficient gene transfer vehicle which can deliver a selected transgene to a selected host cell in vivo or ex vivo even where the organism has neutralizing antibodies to one or more AAV serotypes. In one embodiment, the rAAV and the cells are mixed ex vivo; the infected cells are cultured using conventional methodologies ; and the transduced cells are re-infused into the patient.

These compositions are particularly well suited to gene delivery for therapeutic purposes and for immunization, including inducing protective immunity. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art.

In one embodiment, a method for improving adeno-associated virus (AAV)-mediated gene expression is described. The method involve s gene rating an AAV viral particle comprising a modified packaging insert, wherein said packaging insert comprises a nucleic acid molecule comp rising at least one AAV inverted terminal repeat (ITR) and an exogenous gene sequence under the control of regulatory sequences which control expression of the gene product, wherein said sequences of said nucleic acid molecule are modified to reduce CpG di-nucleotides such that in immune response to the vector is reduced as compared to the unmodified AAV vector without significant reduction in expression of the gene product; and delivering the AAV to a subject intramuscularly. In another embodiment, a regimen for repeat administration of a gene product. The regimen involves delivering to a subject a CpG-depleted adeno-associated viral (AAV) vector. The vector has an AAV capsid having packaged therein a nucleic acid molecule comp rising at least one AAV inverted terminal repeat (ITR) and an exogenous gene sequence under the control of regulatory sequences which control expression of the gene product, wherein said sequences of said nucleic acid molecule are modified to reduce CpG di-nucleotides such that in immune response to the vector is reduced as compared to the unmodified AAV vector without significant reduction in expression of the gene product; and delivering to the subject a second vector comprising the exogenous gene sequence. The second vector may be a second CpG-depleted AAV, which may differ from the first CpG-depleted AA V. Alternatively, a CpG-depleted AA Vas described herein may be used in a regimen using other types of AAV vector, or other viral and non-viral constructs. For example, regimens analogous to those described in EP 1 742 668B1 and WO 2006/078279A2, published 27 Jul. 10, 2006, may be performed using a CpG-depleted AAV of the invention.

### EXAMPLES

The following examples are illustrative only and are not a limitation on the present invention.

### Example 1: In the Absence of TLR9 Signaling, AAVrh32.33nlacZ Muscle Gene Transfer Results in Stable Transgene Expression

The current study assessed the requirement for TLR9 signaling in T cell immuno reactivity and transgene loss in response to AA Vrh32.33. These mechanistic findings were subsequently translated into a modified, CpG-depleted AA Vrh32.33 vector that escapes the adaptive immune response and exhibits stable, long-term transgene expression.

### A. Material and Methods:

1. Mice: C57BL/6 wild type (\VT) mice were ordered from The Jackson Laboratory. Toll-like receptor 9 knockout (TLR9KO) mice were a kind gift from Dr. Phillip Scott (University of Pennsylvania, Philadelphia, Pa.). All mice were housed under specific pathogen-free conditions in the TRL Animal Facility at the University of Pennsylvania. All animal procedure protocols were approved by the Institutional Animal Care and Use Committee of the University of Pennsylvania.
2. Adeno-Associated Viral Vectors (AA V) mediated transduction of the muscle

An AAV8 and AAVrh32.33 pseudotyped vector flanked with AAV2 ITRs encoded a nuclear targeted form of -galactosidase (nLacZ) under the transcriptional control of a CMV-enhanced chicken P-actin (CB) promoter [L. Wang, et al., 1999. Sustained correction of bleeding disorder in hemophilia B mice by gene therapy. Proc Natl Acad Sci USA 96: 3906-3910.] An AAVrh32.33 pseudotyped vector flanked with AAV2 ITRs encoded either a wild type or CpG-depleted cellular targeted form of P-galactosidase (LacZ) (Invivogen). The CpG-depleted promoter and polyA for this construct were also obtained from Invivogen. Over 320 CpGs are present in the wild type Lacz expressing vector (16 CpGs in the inverted terminal repeats (ITRs) and 308 in the transgene); the CpG depleted Lacz vector sequence contains only 16 CpGs loc ated in the ITRs. Outside of the transgene sequence, both vectors are identical in sequence and contain CpG-depleted human elongation factor I-alpha (EIF-a) promoter, CMV enhancer, intron, SV40 3′UTR, and ITRs. AAV vectors were produced by a scaled-down version of a previously described method by triple transfection of vector genome, AAV helper and adenovirus helper plasmids [M. Lock, et al, 2010. Rapid, simple and versatile manufacturing of recombinant adeno-associated viral vectors at scale. Hum Gene Ther 21: 1259-1271]. Purification of vectors involved a single iodixanol step gradient [Zolotukhin, S., et al., 1999]. Recombinant adeno-associated virus purification using novel methods improves infectious titer and yield. Gene Ther 6: 973-985] and subsequent DNAse treatment. Real-time PCR using a primer/probe set corresponding to the poly A region of the vector and linearized plasmid standards determined genome titer (genome copy/mi) of AAV vectors. All vectors used in this study passed the endotoxin assay (threshold 10 EU/mL) using QCL-1000 chromogenic LAL test kit (Cambrex Bio Science). Vectors were produced by Penn Vector Core at the University of Pennsylvania. Mice were injected in the gastroc with 10t1 vector genomes (VG) of AAVin a 50 μl volume.
3. Immunohistochemistry

To examine expression of nuclear P-gal, X-gal staining of snap-frozen liver cryosections was performed according to standard protocols [Bell, P., et al., Histoc hem Cell Biol 124: 77-85.] Representative sections from 4 mice per group were imaged by using brightfield microscopy with a }Ox objective. To analyze major histocompatibility complex class II (MHC II) expression and CD4+/CD8+ infiltrating cell types within the live r, im munostaining and fluorescent microscopy were performed on acetone-fixed cryosections stained with rat anti-CD4 and anti-CD8 antibodies (Ab) from BD Pharmingen and anti-MHC II Ab (Biolegend) as previously described [Mays, L. E., and J. M. Wilson. 2009. J Gene Med 11:1095-1102].
4. MHC Class I Tetramer Staining PE-conjugated MHC class I H2-kb-ICPMYARV tetramer complex was obtained from Beckman Coulter. At kinetic time points after vector injection, tetramer staining was performed on heparinized whole blood cells isolated by retro-orbital bleeds. Cells were co-stained for 30 minutes at room temperature with PE-conjugated tetramer and FITC-conjugated anti-CD8a (Ly-2) Ab (BD Pharmingen) . Red blood cells were lysed and cells were fixed with iTAg MHC tetramer lysing solution supplemented with fix solution (Beckman Coulter) for 15 minutes at room temperature. The cells were then washed three times in PBS and resuspended in lx PBS . Data were gathered with an FCSOO Flow Cytometer (Beckman Coulter) and were analyzed with FlowJo analysis software (Tree Star). In the analysis, lymphocytes were selected on the basis of forward and side scatter characteristics, followed by selection of CD8+ cells, and subsequently the tetramer-positive CD8+ T cell population.
5. ELISPOT Assays for Cytokine-Producing Cells

T cell medium consisted of the following: O:ME M (Cellgro; Mediatech) supplemented with 10% heat-inactivated FBS (Hyclone), 1% penicillin/streptomycin(Cellgro; Mediatech), 1% L-glutamine (Cellgro; Mediatech), 10 mM HEPES buffer (Cellgro; Mediatech), 0.1 mM nonessential amino acids (Invitroge n), 2 mM sodium pyruvate Cellgro; Mediatech) and 10-6 M 2-ME (Cellgro; Mediatech).

Splenocytes were isolated by mechanical dissociation followed by red blood cell (RBC) lysis via hypotonic shock and resuspended at a concentration of 5×10⁶ cells/mL and plated at Sx I 05 cells/well in triplicate on 96-well round-bottom plates. ELISPOT assays were performed according to the manufacturer's instructions (BD Biosciences). T cell medium supplemented with 2 μg/mL of H2-kb-restricted-gal CD8 T cell epitope (ICPMYARV) and H2-kb-restricted AAVrh32.33 capsid epitope (SSYELPYVM) (Mimotopes) was used to stimulate splenocytes. Splenocytes were incubated at 37° C., 5% CO2 for 18 hours. Spots were visualized by addition of 3-Amino-9-Ethylcarbazole (AEC) substrate set (BD Biosciences) and counted using the AID ELISPOT reader system (Cell Technology).
6. RNA Isolation and Quantitative RT-PCR

Gastroc tissue was homogenized in 1 ml TRizol (Life Technologies) and RNA was isolated per the manufacturer's protocol. Total RNA (5 μg) was reverse transcribed using I OxPCR buffer (Roche), 10 mM dNTP, oligo(dt), M-MLV-RT (all from Invitrogen), and RNAsin (Promega). Products were then cleaned with 1:1 phenol/chloroform/isoamyl (25:24 :1) and reprecipitated with 7.5 M NH4Q AC in pure ethanol overnight at −80° C.

Real-time PCR was performed on cDNA using a 7500 Real Time PCR System (Applied Biosystems). Primer binding to DNA was detected by SYBR 2x Master Mix (Applied Biosystems). Relative expression of the gene of interest was expressed as the comparative concentration of the gene product to the GAPDH product. Transcript relative expression of target genes were then expressed as fold induction over mock treated (PBS injected) naive WT mouse gastroc samples. Significance was determined with an unpaired Student t test.
7. Muscle Weight

Muscle weight was determined by weighing the vector injected gastroc and comparing it to the total body weight of the animal on day 60 post-injection.
8. Statistical Analysis

Data were analyzed with GraphPad Prism 4.0c software using unpaired Student t-tests. p values of :5 0.05 were considered statistically significant.

### RESULTS

B. In the Absence of TLR9 Signaling, AAVrh32.33nlacZ Muscle Gene Transfer Results in Stable Transgene Expression.

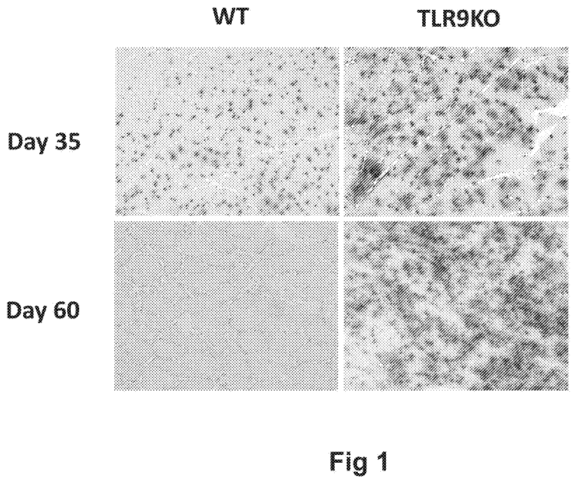

Studies in C57BL/6 mice demonstrate that AAVrh32.33 intramuscular gene transfer induces a robust adaptive immune response towards both capsid and transgene antigen, heavy cellular infiltrate, and a loss of detectable transgene expression [Mays, L. E., et al, 2009. J of Immunol 182: 6051-6060). To evaluate the role of TLR9 signaling in the induction of this adaptive immune response and transgene loss, WT and TLR9KO mice were intramuscularly (I.M.) injected with 1×10¹¹ viral particles of AAVrh32.33 expressing a nuclear-targeted P-gal (nLacZ) reporter gene under the direction of a chicken P-actin promoter. Gastroc tissue was recovered from the WT and TLR9KO mice and p gal expression in the muscle was assessed by X-gal histochemical stain (FIG. 1). WT controls exhibited a complete loss of P-gal positive cells at 60 days post-injection. In contrast, abrogation of TLR9 signaling resulted in stable transgene expression. These results suggest that TLR9 signaling is required for transgene loss following AAVrh32.33 muscle gene transfer.

### C. A Deficiency in TLR9 Signaling Reduces Immunoreactivity Toward AAVrh32.33 Capsid and Transgene Antigen.

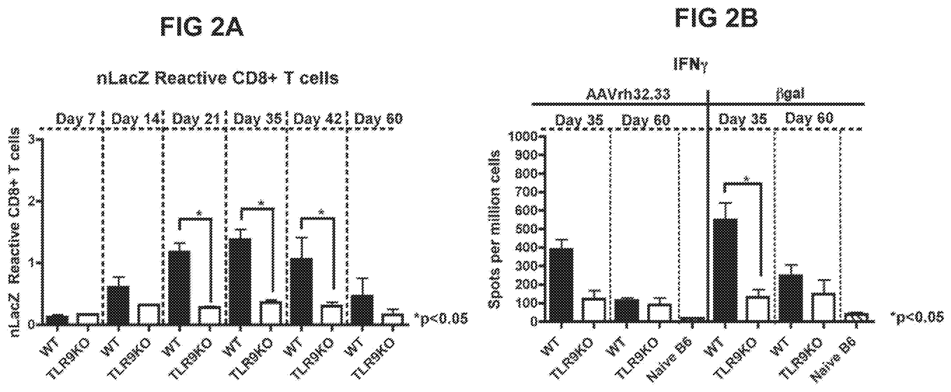

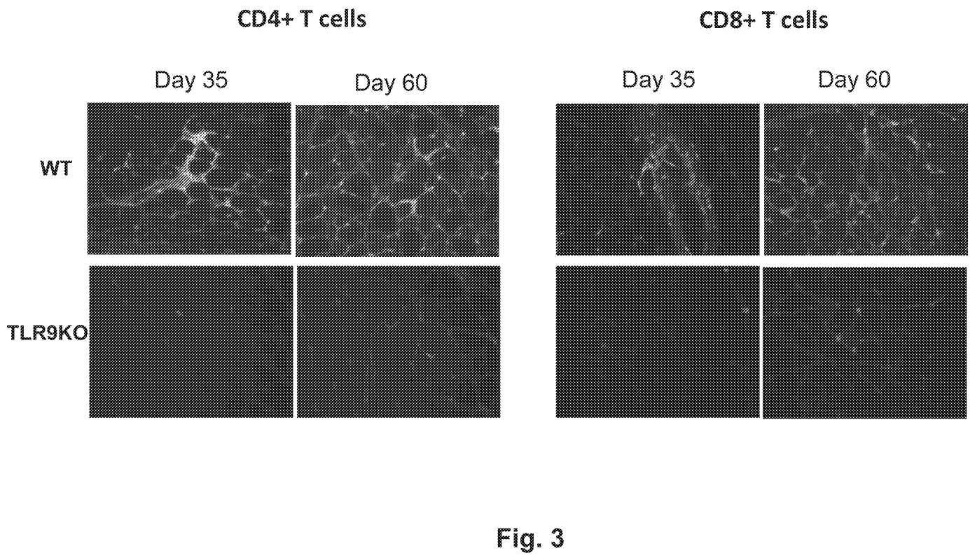

In WT C57BL/6 mice, AAVrh32.33 muscle gene transfer is associated with a robust Th1 response and a significant percentage of nLacZ reactive CDS+ T cells [Mays et al, 2009, J Immunol, cited above]. To investigate the relationship between TLR9 signaling and immunoreactivity, MHC I tetramer stain and ELISPOT assays were used to quantify transgene reactive CDS+ T cells and primed transgene and capsid responsive IFNy-producing cells (FIG. 2). Peripheral blood cells isolated from whole blood were co-stained with a FITC-conjugated anti-CDS Ab and a PE-conjugated H-2Kb-ICPMYARV tetramer to determine the percentage of nLacZ-reactive CDS+ T cells in the total CDS+ T cell population (FIG. 2A). TLR9 deficient mice exhibited a significant reduction in the percentage of nLacZ responsive CDS+ T cell population compared to WT mice.

To determine whether TLR9 signaling regulates AAVrh32.33nLacZ cellular immune responses, we used ELISPOT to quantify the number of in vivo primed capsid and transgene Th 1 (IFNy) responses (FIG. 2B). Elevated capsid and transgene reactive Th1 responses were observed in WT but not TLR9KO vector recipients indicating an abrogation of Th1 responses in the absence of TLR9 signaling. These findings demonstrate the inhibitory effect of TLR9 signaling blockade on T cell effector function following stimulation with AAVrh32.33 capsid and transgene antigens.

D. Minimal Cellular Infiltrate Observed in Muscle Following AAVrh32.33nlacZ I. M. Injection Observed in TLR9 Deficient Mice

Heavy CD4+ and CDS+ cellular infiltrate has been observed in WT C57BL/6 mice following AAVrh32.33 int ramuscular vector transduction [Mays et al, J Immunol, 2009, cited above]. To determine the requirement for TLR9 signaling in the induction of this extensive infiltrate, WT and TLR9KO muscle sections were stained with anti-CD4 and anti-CDS Ab and examined by fluorescent microscopy at 35 and 60 days vector post-administration (FIG. 3). As expected, significant cellular infiltrate was detected in WT mice. In contrast, minimal cellular infiltrate was observed in TLR9KO mice. These results are consistent with a TLR9 dependent mechanism of cellular infiltrate in response to intramuscular AAVrh32.33nLacZ transduction.

E. Enhanced Transgene Expression Observed in the Muscles of TLR9 Deficient Mice Injected with AAV8

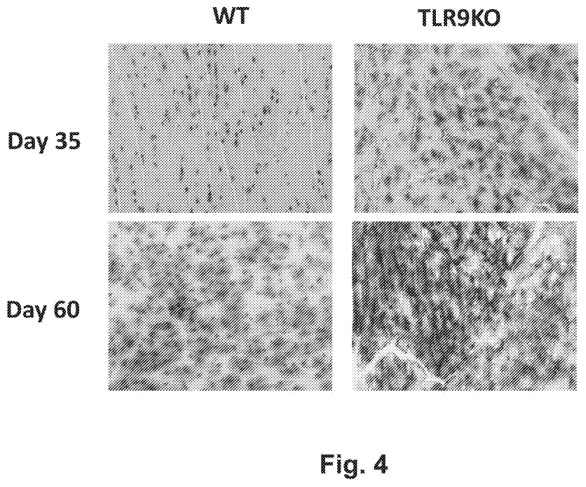

Historically, AAV8 gene delivery to WT C57BLJ6 muscle tissue results in minimal Th1 responses, negligible cellular infiltrate and prolonged transgene expression [Mays et al, J Immunol, 2009, cited above]. To investigate the role of TLR9 detection of AAV8 and its effect, if any, on AAV8 gene expression, WT and TLR9KO mice were injected intramuscularly with 1×10¹¹ GC of AAV8nLacZ. X-gal histochemical stain of muscle sections 35 and 60 days post vector administration (FIG. 4). Enhanced expression is detected in the muscle of TLR9KO mice indicating the detection of AAV8 through this innate immune sensor and an effect on transgene expression even in the case of an AAV vector that induces a minimal immune response.

F. TLR9 Signaling is both Necessary and Sufficient to Upregulate MHC II Expression on a Muscle Tissue

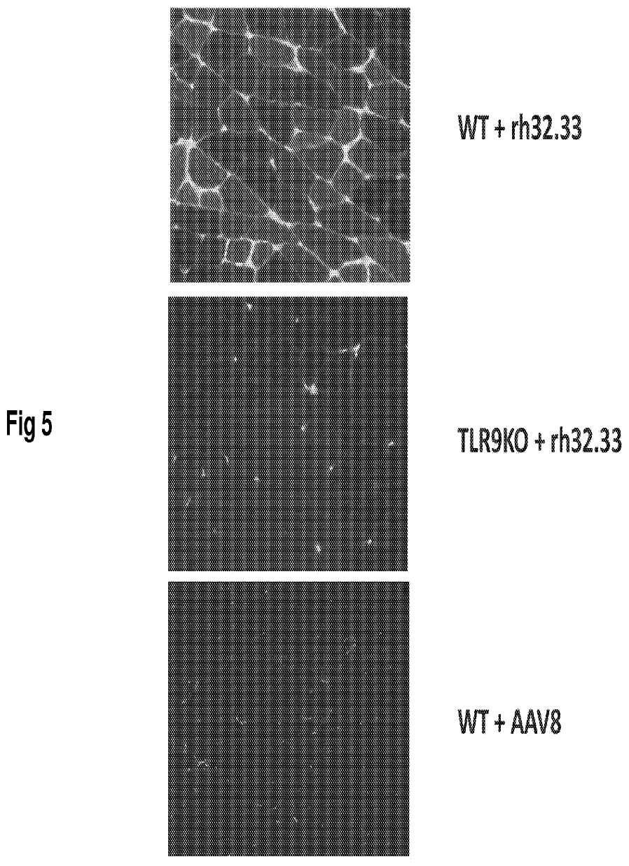

Muscle tissue possesses a unique function as a non-professional antigen presenting cell (APC) that can effectively stimulate both CD4+ and CDS+ T cells to survive, proliferate, and acquire effector function. To assess the ability of AAVrh32.33 to induce :tvfHC II expression on skeletal muscle, gastroc sections from WT and TLR9KO mice that received I. M. injection of 1×1 ou GC of AAV8rh32.33nLacZ or AAV8nLacZ, were stained with an anti-MHC II Ab and examined by fluorescent microscopy 35 days post gene transfer (FIG. 5). WT muscle tissue transduced with AAVrh32.33nLacZ revealed significant upregulation of MHC II on skeletal muscle, which was not present on muscle tissue from TLR9 deficient mice administered AAVrh32.33nLacZ or WT mice that received AAV8. These findings reveal the requirement of TLR9 signaling for MHC II expression on muscle following AAVrh32.33, while AAV8 minimally induces MHC II expression.

### G. AAVrh32.33 Induces Early Innate Immune Gene Transcript Induction in WT, but not TLR9, Deficient Mice

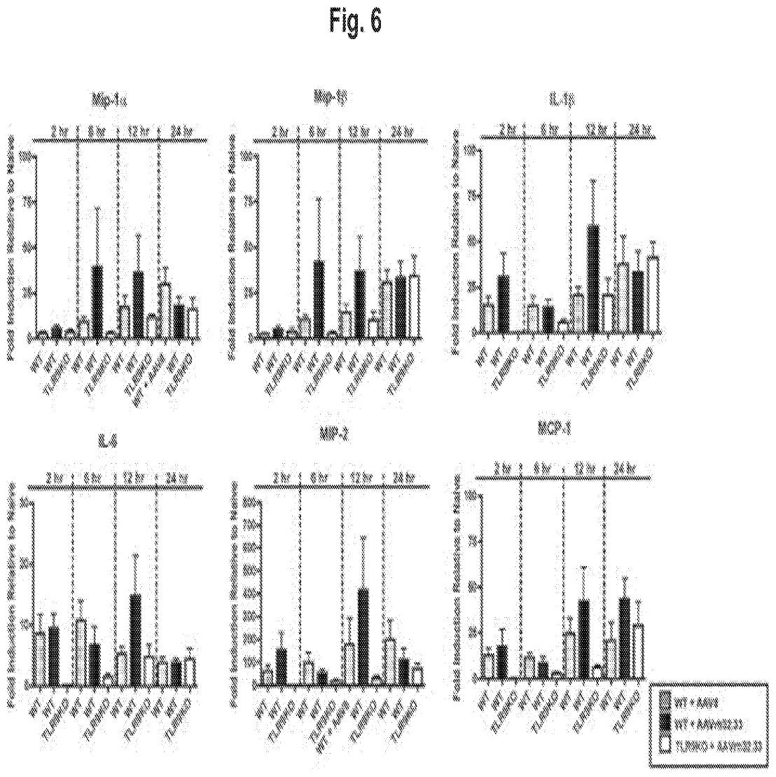

Zais s et al. demon strated that adenovirus vector transduction of human HeLa cells and murine renal epithelium-derived cells induce the expression of numerous chemokines and inflammatory cytokines [Zaiss, A., et al., 2002. J ofVirol 76 (9) 4580-4590].To assay for innate immune gene transcript induction following intramuscular injection of AAVrh32.33 and AAV8 in WT or TLR9 deficient mice, we performed quantitative RT-PCR at early kinetic time points to detect innate chemokine and inflammatory transcript levels following gene transfer (FIG. 6). Transcript levels of MIP-la, MIP-I P, MIP-2, MCP-1, IL-la, and IL-6 were quantified and expressed as fold induction over mock treated (PBS injected) WT mice. A dramatic induction of both chemokine and cytokine transcripts was observed in WT mice transduced with AAVrh32.33, but not in TLR 9K0 mice, or in WT mice administered AAV8. Collectively, our data suggests that TLR9 signaling is both necessary and sufficient to induce innate (FIG. 6) and adaptive (FIG. 2) immune responses toward an immunogenic AAV vector. Our observation that TLR9 deficient AAVrh32.33 vector recipients exhibit minimal immunoreactivity and stable transgene expression led us to hypothesize that CpG depleted AAVrh32.33 vectors would escape immune detection and exhibit long-term transgene expression.

### H. CpG Depleted Vector Generation

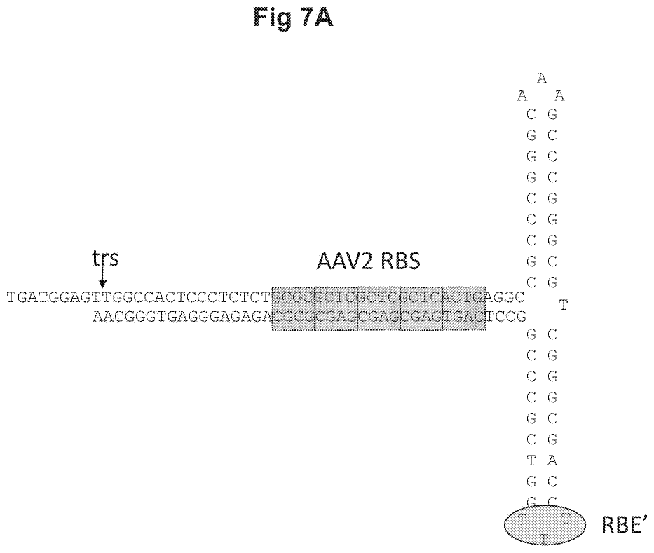

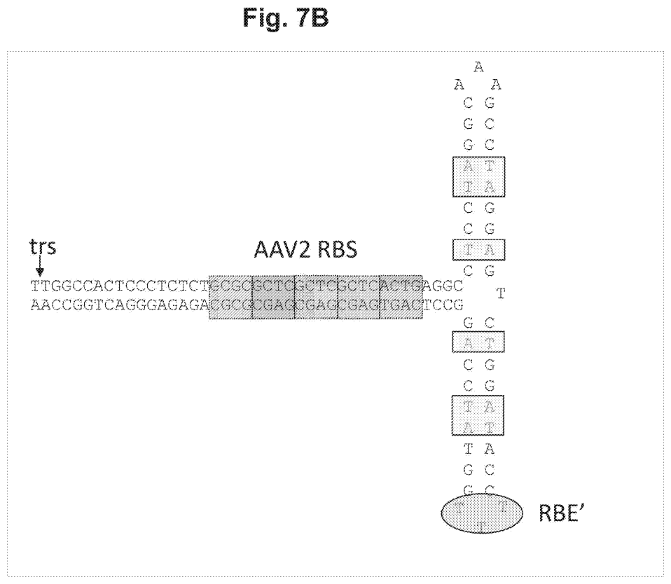

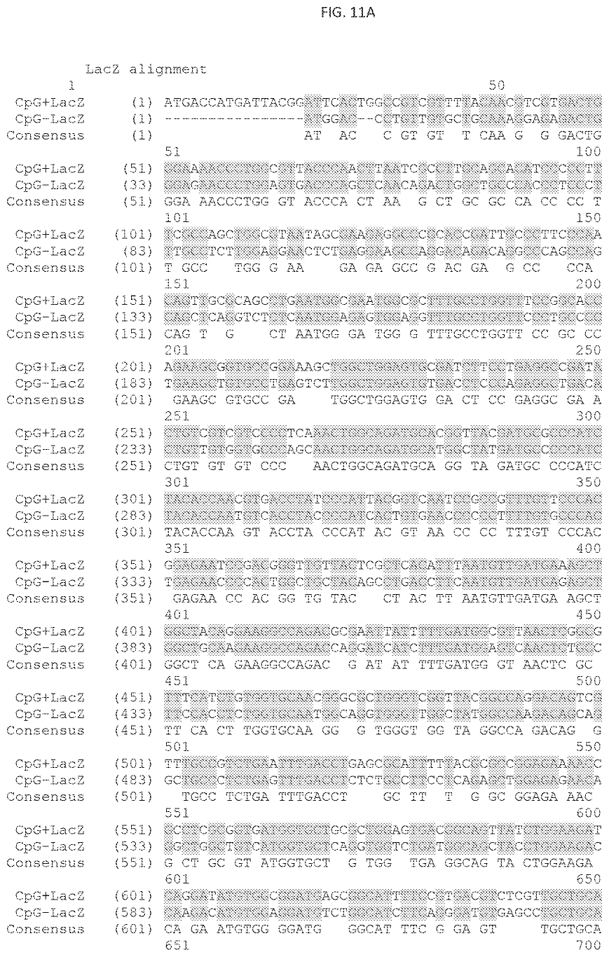

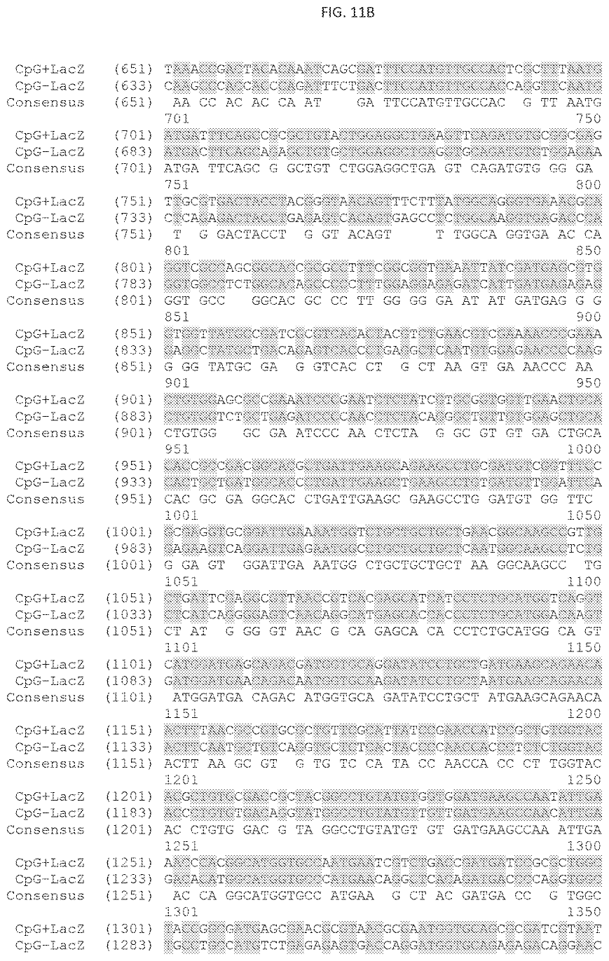

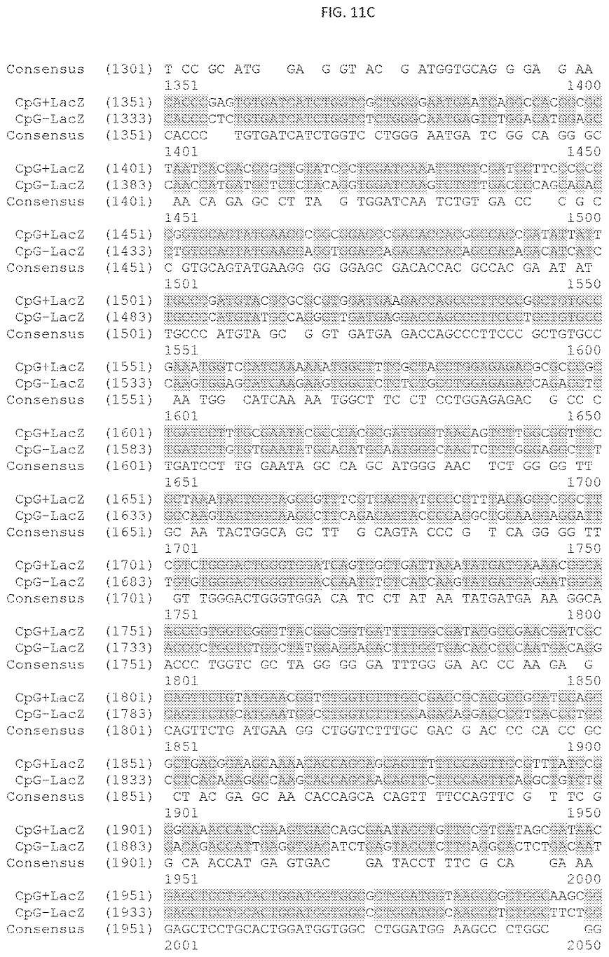

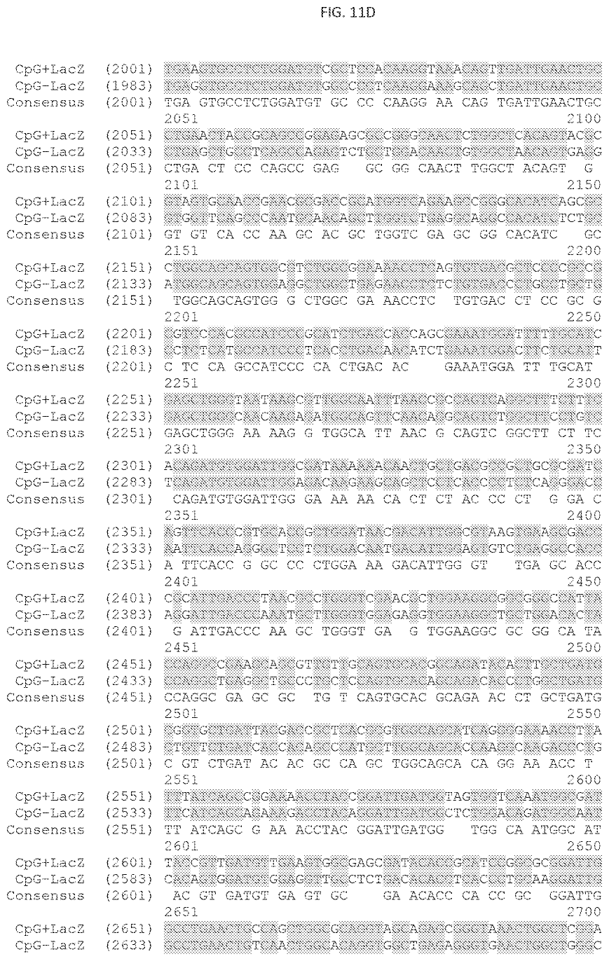

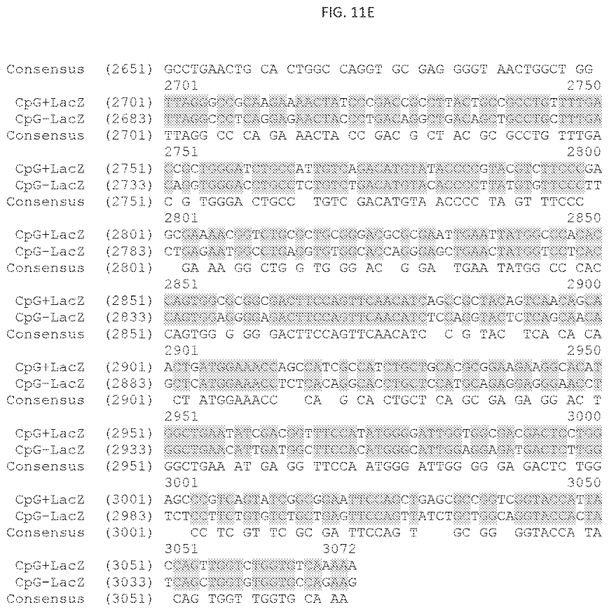

TLR9 acts as an innate immune sensor that specifically recognizes and responds to unmethylated CpG motifs present in ˜7% of microbial genomes compared to ˜1% vertebrate DNA. Systemic delivery of cationic lipid-plasmid DNA (pDNA) vectors that contain CpG motifs stimulate acute inflammatory responses with adverse effects on transgene expression [Yew, N. S., et al, 2002, Mol Ther 5(6): 731-738]. CpG depleted plasmid DNA vectors, on the other hand, exhibit long-term expression and enhanced safety. To determine whether CpG depleted AAVrh32.33 vectors would abrogate the robust cellular immune response and transgene loss we observed in the previous experimen is (FIG. 1-3, 5, 6), we generated a Lacz CpGdepleted vector genome that retained a mere 16 CpGs located in the inverted terminal repeat sequence (FIGS. 7A and 7B; SEQ ID NO: 4 and 5). See, also, sequences provided below. CpGs in the EF1-a promoter, CMV enhancer, Lacz transgene, intron, and SV40 polyA were reduced in the CpG depleted vector (CpG−), while the WT vector (CpG+), which contained the same backbone sequence, retained 16 CpGs in the ITRs as well as 308 CpGs in the Lacz transgene (total=324 CpGs). The sequence of the CpG-modified Lacz transgene is provided in FIG. 11 [SEQ ID NO: 7].

AAV2 ITR alignment document

| SEQ ID NO: 1: | AAV2 ITR | |
|---|---|---|
| SEQ ID NO: 2: | CpG depleted ITR | |
| SEQ ID NO: 3: | Consensus ITR | |
| AAV2 ITR | (1) | AGGAACCCCTAGTGATGGAGTTGGCCACTCCCTCTCTGCGCGCTCGCTCG |
| CpG depleted ITR | (1) | ----------------------GGCCAGTCCCTCTCTGCGCGCTCGCTCG |
| Consensus | (1) | GGCCA TCCCTCTCTGCGCGCTCGCTCG |
| AAV2 ITR | (51) | CTCACTGAGGCCGGGCGACCAAAGGTCGCCCGACGCCCGGGCTTTGCCCG |
| CpG depleted ITR | (29) | CTCACTGAGGCCTGGATACCAAAGGTATCCAGACTCCTAGGCTTTGCCTA |
| Consensus | (51) | CTCACTGAGGCC GG ACCAAAGGT CC GAC CC GGCTTTGCC |
| AAV2 ITR | (101) | GGCGGCCTCAGTGAGCGAGCGAGCGCGCAGAGAGGGAGTGGCCAA----- |
| CpG depleted ITR | (79) | GGAGGCCTCAGTGAGCGAGCGAGCGCGCAGAGAGGGAGTGGCCAACTCCA |
| Consensus | (101) | GG GGCCTCAGTGAGCGAGCGAGCGCGCAGAGAGGGAGTGGCCAA |
| AAV2 ITR | (146) | -------- |
| CpG depleted ITR | (129) | TCACTAGG |
| Consensus | (151) | |

### I. CpG Depleted AAV2/Rh32.33 Vectors Exhibit Stable Transgene Expression

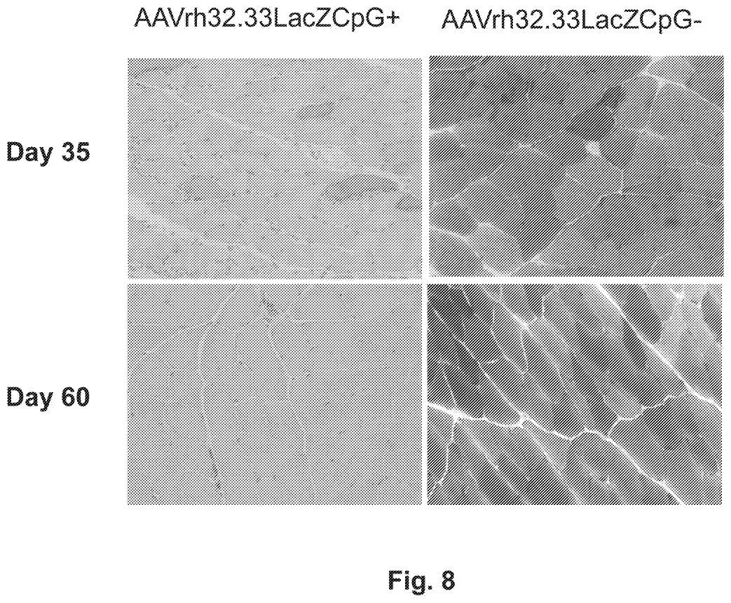

To test our hypothesis that CpG depleted AAVrh32.33 vectors would exhibit prolonged transgene expression, X-gal histochemical stain of gastroc tissues from WT mice injected I. M. with 1E11 GC of AAVrh32.33LacZCpG+ or AAVrh32.33LacZCpG− were assessed (FIG. 8). As expected, X-gal stain of WT AAVrh32.33LacZCpG+ transduced muscle exhibited a steady loss of detectable gal expression. Conversely, the muscle sections from CpG depleted AAVrh32.33LacZ transduced mice displayed robust and stable transgene expression. Hence, the steady loss of Lacz transgene expression following AAVrh32.33LacZ gene transfer is dependent on vector genome CpG motifs.

### J. Evidence of Hypertrophy in AAVrh32.33LacZCpG+ Transduced Muscle

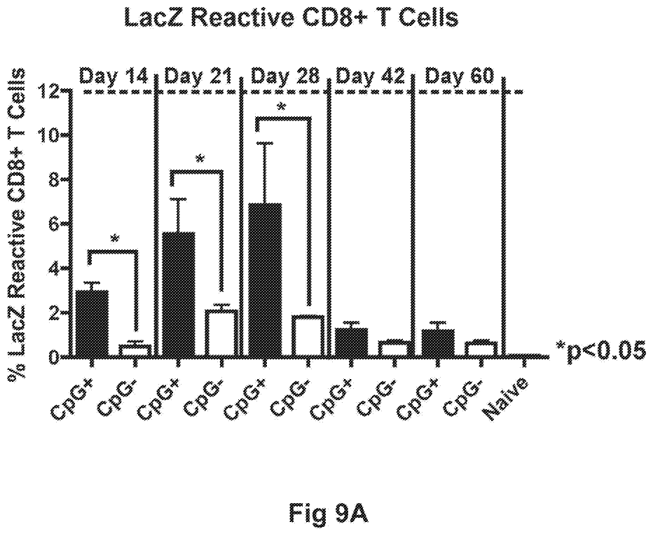

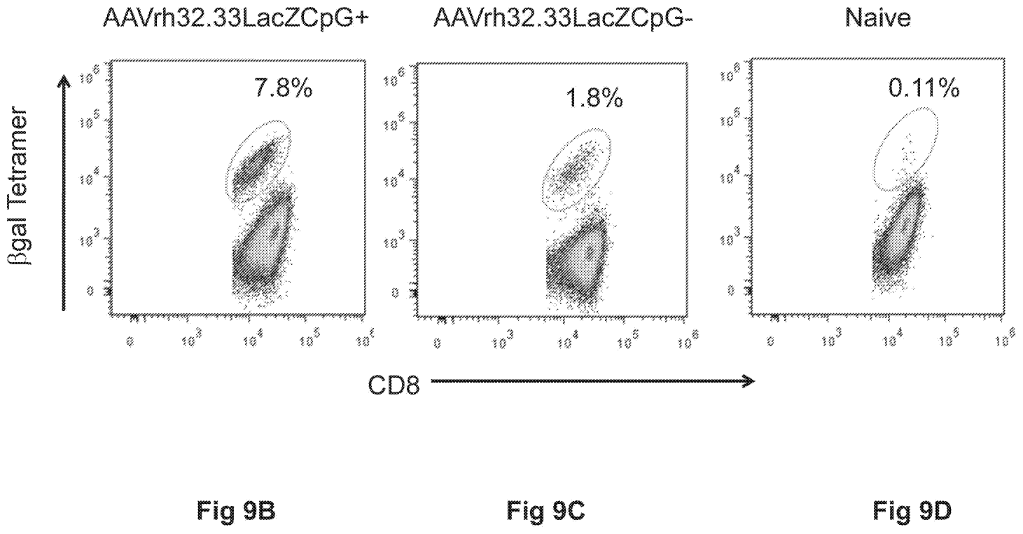

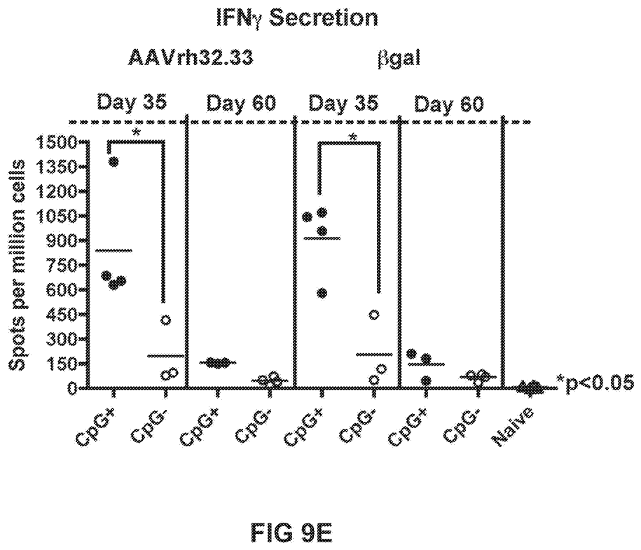

Acute and chronic inflammatory responses are strongly implicated in the induction of a pro-fibrotic environment [Faust, S. M., et al, 2009, J of Immunol 183: 7297-7803]. This inflammatory response stimulates collagen deposition and is commonly associated with the development of cellular hypertrophy. To investigate the impact of TLR9 signaling and the development of hypertrophy following intramuscular AAV gene transfer, gastroc tissue from AAVrh32.33LacZCpG+ or CpG-vector transduced mice was weighed and compared to total body weight (FIG. 9). A statistically significant increase in muscle weight was observed in AAAVrh32.33Cp G+ compared to CpG depleted vector transduced mice. These data reveal the association of AAVrh32.33CpG+ gene transfer and TLR9 stimulated inflammation in the development of muscle hypertrophy.

### K. CpG Depletion Significantly Reduces the Percentage of Lacz Reactive CD8+ T Cells and T Cell Effector Function

Transgene stability observed in the AAVrh32.33LacZCpG-transduced muscle sections (FIG. 8) strongly suggests an abrogated adaptive immune response toward transgene and capsid antigen. To assess the requirement for CpG motifs in the induction of an adaptive immune response toward AAVrh32.33, MHC I tetramer stain and ELISPOT assays were used to quantify transgene reactive CD8+ T cells and primed transgene and capsid responsive I FNy-producing cells as described above (FIG. 9). Mice that received the CpG depleted AAVrh32.33LacZ vector exhibited a significant reduction in the percentage of Lacz responsive CD8+ T cell population compared to control mice (FIG. 9A). Furthermore, a significant decrease in primed transgene and capsid antigen reactive Th1 responses were observed in mice that received the AAVrh32.33CpG− but not CpG+ vector (9B). These findings indicate the ability of a CpG depleted vector to escape immunoreactivity following gene transfer.

L. CpG Depleted AAVrh32.33LacZ Vector Gene Transfer Corresponds with Minimal Cellular Infiltrate and MHC II Expression

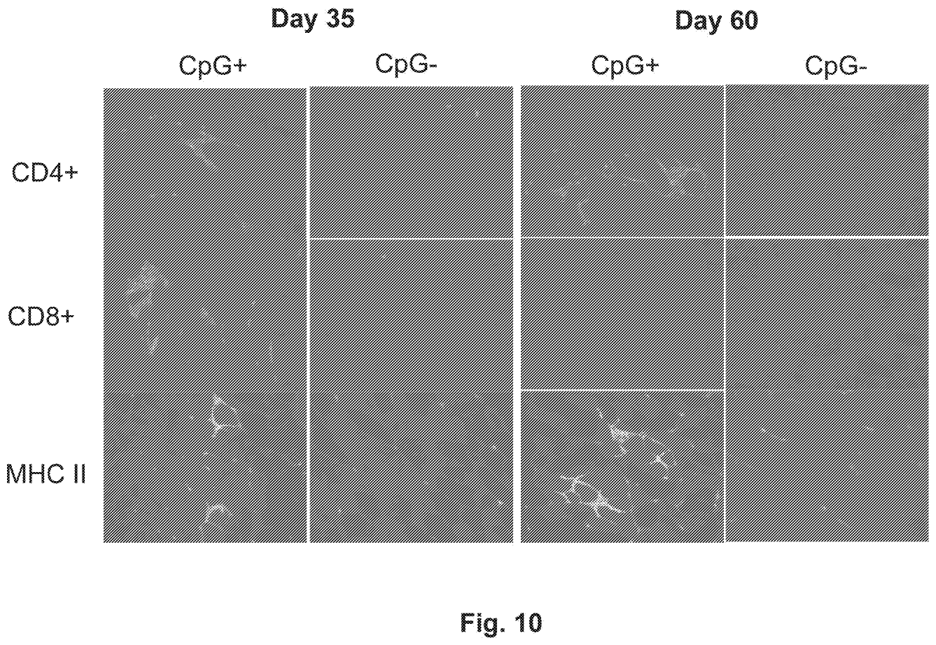

Minimal cellular infiltrate and MHC II expression was revealed in muscle sections in the absence of TLR9 signaling following AAVrh32.33nLacZ gene transfer (FIGS. 3 and 5). These data are consistent with a TLR9 dependent mechanism of cellular infiltrate and MHC II skeletal muscle gene induction in response to the immunogenic AAV vector. If TLR9 signaling is both necessary and significant for these phenomenon, it is reasonable to suggest that CpG depleted AAVrh32.33LacZ vectors should exhibit similar histological findings to TLR9KO mice. To test this hypothesis, 1×10¹¹ GC of AAVrh32.33LacZCpG+ and CpG-vectors were injected intramuscularly and muscle sections were stained with anti-CD4, anti-CD8 and anti-MHC II Ab (FIG. 10). Consistent with our theory, muscle transduced with AAVrh32.33CpG-vector revealed minimal cellular infiltrate and MHC II expression compared to AAVrh32.33CpG+ transduced muscle. These data reveal the ability of CpG depleted AAV vectors to establish long-term transgene expression (FIG. 8), evade immune activation (FIG. 9), prevent the infiltration of effector T cells, and subvert the induction of skeletal muscle MHC II expression (FIG. 10).

### Example 2

To measure Lacz expression ofRhCpG+ and CpG− constructs, HeLa cells were transfected with CpG+ and CpG− AAV expression plasmids. Four days post transfection cells were assayed for-galactosidase activity using the Mammalian 3-galactosidase assay kit as instructed for adherent cells. Absorbance was measured at 405 nm on a TECAN Infinite M1000 PRO plate reader. CpG+ and CpG− AAV vector constructs exhibited comparable Lacz plasmid expression. These data demonstrate that transgene loss in the skeletal muscle of RhCpG+ gene transferred is not due to differential 3-gal expression levels at the plasmid level.

To assess transgene stability and cellular infiltrate at an early kinetic time point, mice were injected intramuscularly with 1×10¹¹ GC of RhCpG+ or RhCpG− vector. 14 days post vector injection, gastrocnemius was harvested and skeletal muscle cryosections were stained with CD4 or CD8 monoclonal antibody (MAb) as well as X-gal. Stable transgene expression and minimal cellular infiltrate was observed in animals that receive RhCpG-vector. This indicates that reduced cellular immunity is observed as early as day 14 post vector transduction.

To examine adaptive immunity toward AAV associated antigen, splenocytes were harvested 7 and 14 days post intramuscular injection of RhCpG+ or RhCpG−. ELISPOT analysis was performed to quantify spots oflFNy per million cells. Similar Th1 responses were observed at the early time point. In contrast, a robust Th1 response toward transgene antigen is observed only in the RhCpG+ vector transduced animals at day 14. These data demonstrate suppressed Th1 responses following RhCpG− administration at an early kinetic time point.

To determine whether tolerance is induced toward the-gal transgene following RhCpG− gene transfer, mice were injected with 1×10¹¹ GC of RhCpG+ or RhCpG− in the right gastrocnemius muscle. 60 days post primary administ ration, mice were injected in the contralateral muscle with 1×10¹¹ GC of RhCpG+. Both right and left gastrocnemius tissue was harvested and muscle cryosections were stained with CD4/CD8 MAb as well as X-gal 35 days post-secondary injection. Transgene expression and minimal cellular infiltrate was exhibited in the right gastrocnemius of the RhCpG− gene transferred mice indicating a localized tolerance in the skeletal muscle even following an immunogenic vector administration.

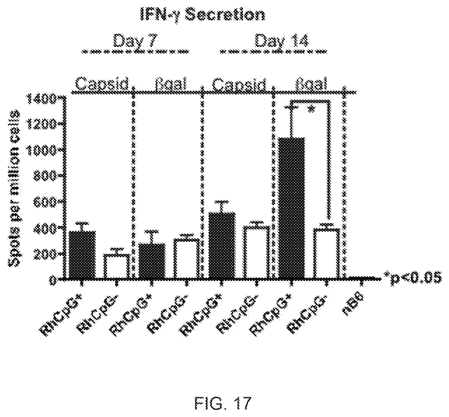

To analyze the Th1 response in the animals that receive a secondary injection of RhCpG+, mice were injected with RhCpG+ or RhCpG− in the right gastrocnemius muscle and 60 days post primary administration, injected in the contralateral muscle with RhCpG+ (FIG. 17). Splenocytes were harvested 35 days post-secondary administrations and ELISPOT was performed to quantify spots of IFNy per million cells. Interestingly, comparable Th1 responses in mice gene transferred initially with RhCpG+ or RhCpG− vector and readministered RhCpG+ vector, even in the presence of localized tolerance in the skeletal muscle of RhCpG− transduced animals. The se data suggests that even in the presence of transgene reactive T cells, a factor localized in the skeletal muscle of mice administered the CpG depleted vector prevents the extinguishment of-gal expression.

All publications cited in this specification are incorporated herein by reference, is U.S. Patent Application No. 61/785,368, filed Mar. 14, 2013. While the invention has been described with reference to a particularly preferred embodiment, it will be appreciated that modifications can be made without departing from the spirit of the invention. Such modifications are intended to fall within the scope of the appended claims.

1-21. (canceled)

22. A method for producing CpG-depleted adeno-associated virus (AAV) vector, comprising
(a) producing a CpG-depleted expression cassette comprising an exogenous transgene encoding a gene product operably linked to a regulatory sequence and flanked by a 5′ AAV inverted terminal repeat (ITR) and a 3′ AAV ITR,
(b) delivering the CpG-depleted expression cassette to a host cell that comprises a nucleic acid sequence encoding AAV capsid proteins and a nucleic acid sequence encoding AAV replication proteins,
(c) culturing the host cell under conditions suitable to permit expression and production of AAV viral particles comprising a capsid produced from the AAV capsid proteins and the CpG-depleted expression cassette packaged within the capsid, and
(d) purifying and isolating AAV viral particles from the cell culture, thereby producing CpG-depleted adeno-associated virus (AAV) vectors.

23. The method of claim 22, wherein the 5′ AAV ITR and 3′ AAV ITR are genetically modified to reduce CpG di-nucleotides.

24. The method of claim 23, wherein the 5′ AAV ITR and 3′ AAV ITR are genetically modified to be CpG-free.

25. The method of claim 22, wherein the transgene is genetically modified to reduce CpG di-nucleotides.

26. The method of claim 25, wherein the transgene is genetically modified to be CpG-free.

27. The method of claim 22, wherein the regulatory sequence is genetically modified to reduce CpG di-nucleotides.

28. The method of claim 22, wherein the regulatory sequence is genetically modified to be CpG-free.

29. The method of claim 22, wherein the 5′-ITR, 3′-ITR, and rep sequences are from the same AAV serotype.

30. The method of claim 22, wherein the nucleic acid sequences encoding the capsid and replication proteins are from the same AAV serotype.

31. The method of claim 22, wherein the nucleic acid sequences encoding the capsid and replication proteins are from a different AAV serotype.

32. The method of claim 22, wherein the host cell comprises a prokaryotic cell.

33. The method of claim 22, wherein the host cell comprises a eukaryotic cell.

34. The method of claim 33, wherein the mammalian cell comprises an insect cell.

35. The method of claim 33, wherein the mammalian cell comprises a yeast cell.

36. The method of claim 33, wherein the mammalian cell comprises an A549, WEHI, 3T3, 10T1/2, BHK, MDCK, COS1, COS7, BSSC1, BSC40, BMT10, VERO, WI38, HeLa, 293, a C2C12, L, HT1080, or HepG2 cell.

37. The method of claim 33, wherein the mammalian cell comprises a 293 cell which expresses functional adenoviral E1.

38. The method of claim 33, wherein the mammalian cell comprises a hepatocyte, a primary fibroblast, or a myoblast cell.

39. The method of claim 33, wherein the mammalian cell is derived from a human, monkey, mouse, rat, rabbit, or hamster cell.


SEQUENCE LISTING (14 sequences)

SEQ ID NO: 1 - DNA, 145 bp, adeno-associated virus 2
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 136 bp, synthetic construct
  misc_feature 1..136: note = Synthetic (CPG Depleted ITR)
  source 1..136: mol_type = other DNA
ggccagtccctctctgcgcgctcgctcgctcactgaggcctggataccaaaggtatccagactcctaggctttgcctaggaggcctcagtgagcgagcgagcgcgcagagagggagtggccaactccatcactagg

SEQ ID NO: 3 - DNA, 123 bp, synthetic construct
  misc_feature 1..123: note = Synthetic sequence (consensus AAV ITR)
  misc_feature 6: note = n is a, c, g, or t
  misc_feature 41: note = n is a, c, g, or t
  misc_feature 44..45: note = n is a, c, g, or t
  misc_feature 55..56: note = n is a, c, g, or t
  misc_feature 59: note = n is a, c, g, or t
  misc_feature 63: note = n is a, c, g, or t
  misc_feature 66..67: note = n is a, c, g, or t
  misc_feature 77..78: note = n is a, c, g, or t
  misc_feature 81: note = n is a, c, g, or t
  source 1..123: mol_type = other DNA
ggccantccctctctgcgcgctcgctcgctcactgaggccnggnnaccaaaggtnnccngacnccnnggctttgccnnggnggcctcagtgagcgagcgagcgcgcagagagggagtggccaa

SEQ ID NO: 4 - DNA, 138 bp, adeno-associated virus 2
  source 1..138: mol_type = other DNA
tgatggagttggccactccctctctgcgcgctcgctcgctcactgaggccggccccggggcaaagcccgggcgtcgggcgacctttggtcgccccggcctcagtgagcgagcgagcgcgcagagagggagtggggcaa

SEQ ID NO: 5 - DNA, 125 bp, synthetic construct
  misc_feature 1..125: note = Synthetic Sequence (AAV ITRs with GpG outside of RBS)
  source 1..125: mol_type = other DNA
ttggccactccctctctgcgcgctcgctcgctcactgaggcctcctaggcaaagcctaggagtctggagacctttggtatccaggcctcagtgagcgagcgagcgcgcagagagggactggccaa

SEQ ID NO: 6 - DNA, 3072 bp, Escherichia coli
  source 1..3072: mol_type = other DNA
atgaccatgattacggattcactggccgtcgttttacaacgtcgtgactgggaaaaccctggcgttacccaacttaatcgccttgcagcacatccccctttcgccagctggcgtaatagcgaagaggcccgcaccgattgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcggtaccattaccagttggtctggtgtcaaaaa

SEQ ID NO: 7 - DNA, 3056 bp, synthetic construct
  misc_feature 1..3056: note = Synthetic sequence (CpG-depleted LacZ)
  misc_feature 7..8: note = n is a, c, g, or t
  source 1..3056: mol_type = other DNA
atggacnncctgttgtgctgcaaaggagagactgggagaaccctggagtgacccagctcaacagactggctgcccaccctccctttgcctcttggaggaactctgaggaagccaggacagacaggcccagccagcagctcaggtctctcaatggagagtggaggtttgcctggttccctgcccctgaagctgtgcctgagtcttggctggagtgtgacctcccagaggctgacactgttgtggtgcccagcaactggcagatgcatggctatgatgcccccatctacaccaatgtcacctaccccatcactgtgaaccccccttttgtgcccactgagaaccccactggctgctacagcctgaccttcaatgttgatgagagctggctgcaagaaggccagaccaggatcatctttgatggagtcaactctgccttccacctctggtgcaatggcaggtgggttggctatggccaagacagcaggctgccctctgagtttgacctctctgccttcctcagagctggagagaacaggctggctgtcatggtgctcaggtggtctgatggcagctacctggaagaccaagacatgtggaggatgtctggcatcttcagggatgtgagcctgctgcacaagcccaccacccagatttctgacttccatgttgccaccaggttcaatgatgacttcagcagagctgtgctggaggctgaggtgcagatgtgtggagaactcagagactacctgagagtcacagtgagcctctggcaaggtgagacccaggtggcctctggcacagccccctttggaggagagatcattgatgagagaggaggctatgctgacagagtcaccctgaggctcaatgtggagaaccccaagctgtggtctgctgagatccccaacctctacagggctgttgtggagctgcacactgctgatggcaccctgattgaagctgaagcctgtgatgttggattcagagaagtcaggattgagaatggcctgctgctgctcaatggcaagcctctgctcatcaggggagtcaacaggcatgagcaccaccctctgcatggacaagtgatggatgaacagacaatggtgcaagatatcctgctaatgaagcagaacaacttcaatgctgtcaggtgctctcactaccccaaccaccctctctggtacaccctgtgtgacaggtatggcctgtatgttgttgatgaagccaacattgagacacatggcatggtgcccatgaacaggctcacagatgaccccaggtggctgcctgccatgtctgagagagtgaccaggatggtgcagagagacaggaaccacccctctgtgatcatctggtctctgggcaatgagtctggacatggagccaaccatgatgctctctacaggtggatcaagtctgttgaccccagcagacctgtgcagtatgaaggaggtggagcagacaccacagccacagacatcatctgccccatgtatgccagggttgatgaggaccagcccttccctgctgtgcccaagtggagcatcaagaagtggctctctctgcctggagagaccagacctctgatcctgtgtgaatatgcacatgcaatgggcaactctctgggaggctttgccaagtactggcaagccttcagacagtaccccaggctgcaaggaggatttgtgtgggactgggtggaccaatctctcatcaagtatgatgagaatggcaacccctggtctgcctatggaggagactttggtgacacccccaatgacaggcagttctgcatgaatggcctggtctttgcagacaggacccctcaccctgccctcacagaggccaagcaccagcaacagttcttccagttcaggctgtctggacagaccattgaggtgacatctgagtacctcttcaggcactctgacaatgagctcctgcactggatggtggccctggatggcaagcctctggcttctggtgaggtgcctctggatgtggcccctcaaggaaagcagctgattgaactgcctgagctgcctcagccagagtctgctggacaactgtggctaacagtgagggtggttcagcccaatgcaacagcttggtctgaggcaggccacatctctgcatggcagcagtggaggctggctgagaacctctctgtgaccctgcctgctgcctctcatgccatccctcacctgacaacatctgaaatggacttctgcattgagctgggcaacaagagatggcagttcaacaggcagtctggcttcctgtctcagatgtggattggagacaagaagcagctcctcacccctctcagggaccaattcaccagggctcctctggacaatgacattggagtgtctgaggccaccaggattgacccaaatgcttgggtggagaggtggaaggctgctggacactaccaggctgaggctgccctgctccagtgcacagcagacaccctggctgatgctgttctgatcaccacagcccatgcttggcagcaccaaggcaagaccctgttcatcagcagaaagacctacaggattgatggctctggacagatggcaatcacagtggatgtggaggttgcctctgacacacctcaccctgcaaggattggcctgaactgtcaactggcacaggtggctgagagggtgaactggctgggcttaggccctcaggagaactaccctgacaggctgacagctgcctgctttgacaggtgggacctgcctctgtctgacatgtacaccccttatgtgttcccttctgagaatggcctgaggtgtggcaccagggagctgaactatggtcctcaccagtggaggggagacttccagttcaacatctccaggtactctcagcaacagctcatggaaacctctcacaggcacctgctccatgcagaggagggaacctggctgaacattgatggcttccacatgggcattggaggagatgactcttggtctccttctgtgtctgctgagttccagttatctgctggcaggtaccactatcagctggtgtggtgccagaag

SEQ ID NO: 8 - DNA, 1856 bp, synthetic construct
  misc_feature 1..1856: note = Synthetic sequence (CpG-modified human TnT intron w S10001 exons
  source 1..1856: mol_type = other DNA
gaattctgtacagtagcttccaccatgggctctgagctggagacagctatggagaccctgatcaatgtgttccatgcccactctggcaaggagggagacaagtacaagctgagcaagaaggagctgaaggagctgctccagacagagctgtctggcttcctggatgcccagaaggtgagtgccatgctgtccccagcccagcatctccatctcacactcctcctggttcactgggtcctggtaatactggtccctgggtccctgtccctattccccaacagcccccttcagctcctgcatctgcccctgctgcctggccttcagcagtgttccatgtccacccattgaccactgcttgctggaagtgtctgagagctcctggggctgagcagagacactttcctggtgttccaaccctgggggtctccaacacttgaggcagcagctcagtgatctgagctggttacaaggacctggatgcaccaagccaagcagctcagtgatctgagctggttacaaggaccaggatgcaccaagccaggacccccagtggaaggggagtgctgccaacagagaggtgcttctccccacatacacccaaggtcctggtgtgggcacaattaggctgagcctcaagctcacagtctttggagtcctatgtgcactaatgagggtcttaggtgaacagacactggcaaggaaatggcttagaggacactgatgctgcataccatgagcttagacctgggcccagtccttccttacccaccacccccagcccctggtccctagggcctctttgctacctaagggaagaacttcagcttcccctggaaggtctcctttgctgctcctgccaaaccactcctccctgggcaagaagcccctgctgggctggcttggctacgggtactcccacctcccaataggagagaggctgtattgcctggtgacagtggcatggactttggagccataatgcctgggttgaatttctacctgtgcccctcactggctgtgtgacattggcagagttagtccactgttccttgcctccatttccacatggtaacactacaatatacttcagagggtgattgtgagggttacagagataatactaattgttattattgctatagtgttccaaccactgttccaagcatgtcccatgggtgctcagaagagcctggcacccactaaatgctcagcatgtcagccattgttatggcctctctagtcctgtgccttccacttttttctctttttttggttccacactgaactctgcaccaagccaaaggacacagatttgccaaactttggggcagcacctgggtggtgcatggggatgctactgctcaaagggcacagcttcctgggatggtgggcagctgggcatgggtgccccagaggggtctggggctgggctgctaggagggctccatgacacagcctccagctttgtgcccagctctcagaggcccttcttatgggactctcatatcctgaacctattatggccctgggaccccacagtgggaggcccatgaggcatcctggaaggcttctccttggcttctgcctgtggtacaaggcccctcctgtcccttaactatcctacccctcctactcttccatgctcctccttctcctcctgcactgctgcactcagcccccttctccccatcccctggccacccctgaccatcctcccttgctctttgtccttccccttttcttgcaggatgtggatgctgtggacaaggtgatgaaggagctggatgagaatggagatggcctgcaacaacttcttctgggagaacagctgatggtctgctagctctagaggatcc

SEQ ID NO: 9 - DNA, 1746 bp, Homo sapiens
  source 1..1746: mol_type = other DNA
gagaaggccaaggagctgtggcagagcatctataacttggaggcagagaagttcgacctgcaggagaagttcaagcagcagaaatatgaggtgggccgccatgctgtccccgcccagcatctccatctcacactcctcctggttcactgggtccggtaatactgctgcaggtccctgggtccctgtccctattccccaacagcccccttcagctcctgcatctgcccctgctgcctggccttcagcagtgttccatgtccaccgcattgacgactgcttgctggaagtgtctgagagctcgctggggctgagcagagacactttcctggtgttccaaccctgggggtctccaacacgttgaggcagcagctcagtgatctgagctggttacaaggaccaggatgcaccaagccaggacccccagtggaaggggagtgctgccaacagagaggtgcttctccccacatacacccaaggtcctggtgtgggcacaattaggctgagcctcaagctcacagtctttcggagtcctatgtgcactaatgagggtcttaggtgaacagacaccaggcaaggaaatggcttagaggacactgatgctgcataccggagcttagacctgggcgccagtccttccttacccaccacccccgagccccggtcccagggcctctttgctacctaagggaagaattcagcttcccctggaaggtctcctttgctgctcctgccaaaccactcctccctgggcaagaagcccctcgctgggcaggctcaggctgcagtggctacagggtactcccacctcccaatacggagagaggctgtattgcctggtgacagtggcatggactttggagccataatgcctgggttgaatttctacctgtgcccctcactggctgtgtgacattgggtgagttagtccactgttccttgcctccatttccacaggtaacactacaatatacttcagagggtgattgtgagggttacagagataatactaattgttattattgctatagtgttccaaccactgttccaagcatgtcccatgtattaacttactatgccctcatagcagccctatgggttcatatctgggaaggtgctcaggacagagcctggcacccactaaatgctcagcaggtgtcagccattgttatggcctctctagtcctgtgccttccacttttttctctttttttggttccacactgaactctgcaccggccaacaggacacagatttgccaaactttggggcagcaccctgcaggggtggtgcatggggatgctactgctcaaagggcacagcttccgggatggtgggcagctgggcaggggtgccccagaggggtctggggctgggctgctaggagggctccatgacacagcctccagctttgtgcccagctctcagaggcccttcttatgggactctcatatcctgaacctattatggccctgggaccccacagtgggaggcccatgaggcatcctggaagcttctccttggcttctgcctgtggtacacgggcccctcctgacccttaactatcctaacccctcctactcttccatgctcctccttctcctcctgcactgctgcactcagcccccttctccccatccccaggccacctgggacctgagccagtcagctccagcgttgctctttgtccttcccacttttcttgcagatcaatgttctccgaaacaggatcaacgataaccagaaagt

SEQ ID NO: 10 - DNA, 1700 bp, Photinus pyralis
  source 1..1700: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgccatgggcatcagccagcccacagtggtatttgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtg

SEQ ID NO: 11 - DNA, 1650 bp, synthetic construct
  misc_feature 1..1650: note = Synthetic sequence (cpg-depleted firefly luciferase)
  source 1..1650: mol_type = other DNA
atggaagatgccaaaaacattaagaagggcccagctccattctacccactggaagatgggacagctggagagcagctgcacaaagccatgaagagatatgccctggtgcctggcaccattgcctttacagatgcacatattgaggtggacattacctatgctgagtactttgagatgtctgttagactggcagaagctatgaagagatatgggctgaatacaaaccataggattgtggtgtgctctgagaatagcttgcagttcttcatgcctgtgttgggtgccctgttcattggtgtggctgtggccccagctaatgacatctacaatgagagagagctgctgaacagcatgggcatcagccagcccacagtggtatttgtgagcaagaaagggctgcaaaagatcctcaatgtgcaaaagaagctacccatcatacaaaagatcatcatcatggatagcaagacagactaccagggcttccaaagcatgtatacctttgtgacttcccatttgccacctggcttcaatgagtatgactttgtgcctgagagctttgacagagacaaaaccattgccctgatcatgaacagtagtggcagtacaggattgcccaagggagtagccctacctcacagaacagcttgtgtcagattcagtcatgccagggaccccatctttggcaaccagatcatccctgacacagctatcctctctgtggtgccatttcatcatggctttggcatgttcaccaccctgggctacttgatctgtggctttagggtggtgctcatgtatagatttgaggaggagctattcttgaggagcttgcaagactataagattcaatctgccctgctggtgcccacactatttagcttctttgctaagagcactctcattgacaagtatgacctaagcaacttgcatgagattgcctctggaggggcccctctcagcaaggaggtaggtgaggctgtggccaaaaggttccacctaccaggcatcaggcagggctatggcctgacagaaacaacctctgccattctgatcacccctgaaggggatgacaagcctggagcagtaggcaaggtggtgcccttctttgaggctaaggtggtggacttggacacaggtaagacactgggtgtgaaccagagaggagagctgtgtgtcaggggccccatgatcatgtctggctatgttaacaaccctgaggctacaaatgctctcattgacaaggatggctggctgcactctggagacattgcctactgggatgaggatgagcacttcttcattgtggacaggctgaagagcctgatcaaatacaagggctaccaggtagccccagctgaactggagagcatcctgctgcaacaccccaacatctttgatgctggggtggctggcctgcctgatgatgatgcaggagagctgccagcagcagttgtggtgctggaacatggtaaaaccatgacagagaaggagattgtggactatgtggccagccaggttacaacagccaagaagctgagaggtggtgttgtgtttgtggatgaggtgcctaaaggactgacaggcaagttggatgccagaaagatcagagagattctcattaaggccaagaagggaggcaagattgctgtg

SEQ ID NO: 12 - DNA, 5978 bp, synthetic construct
  misc_feature 1..5978: note = Synthetic sequence (CpG-modified vector)
  source 1..5978: mol_type = other DNA
gaattcttggccagtccctctctgcgcgctcgctcgctcactgaggcctggataccaaaggtatccagactcctaggctttgcctaggaggcctcagtgagcgagcgagcgcgcagagagggagtggccaactccatcactaggggttcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggttggtgtacagtagcttccaccatgggctctgagctggagacagctatggagaccctgatcaatgtgttccatgcccactctggcaaggagggagacaagtacaagctgagcaagaaggagctgaaggagctgctccagacagagctgtctggcttcctggatgcccagaaggtgagtgccatgctgtccccagcccagcatctccatctcacactcctcctggttcactgggtcctggtaatactggtccctgggtccctgtccctattccccaacagcccccttcagctcctgcatctgcccctgctgcctggccttcagcagtgttccatgtccacccattgaccactgcttgctggaagtgtctgagagctcctggggctgagcagagacactttcctggtgttccaaccctgggggtctccaacacttgaggcagcagctcagtgatctgagctggttacaaggacctggatgcaccaagccaaggacccccagtggaaggggagtgctgccaacagagaggtgcttctccccacatacacccaaggtcctggtgtgggcacaattaggctgagcctcaagctcacagtctttggagtcctatgtgcactaatgagggtcttaggtgaacagacactggcaaggaaatggcttagaggacactgatgctgcataccatgagcttagacctgggcccagtccttccttacccaccacccccagcccctggtccctagggcctctttgctacctaagggaagaacttcagcttcccctggaaggtctcctttgctgctcctgccaaaccactcctccctgggcaagaagcccctgctgggctggcttggctagggtactcccacctcccaataggagagaggctgtattgcctggtgacagtggcatggactttggagccataatgcctgggttgaatttctacctgtgcccctcactggctgtgtgacattggcagagttagtccactgttccttgcctccatttccacatggtaacactacaatatacttcagagggtgattgtgcagggttacagagataatactaattgttattattgctatagtgttccaaccactgttccaagcatgtcccatgtattaacttactatgccctcatagcagccctatgggttcatatctgggaaggtgctcagaagagcctggcacccactaaatgctcagcatgtcagccattgttatggcctctctagtcctgtgccttccacttttttctctttttttggttccacactgaactctgcaccaagccaaaggacacagatttgccaaactttggggcagcacctgggtggtgcatggggatgctactgctcaaagggcacagcttcctgggatggtgggcagctgggcatgggtgccccagaggggtctggggctgggctgctaggagggctccatgacacagcctccagctttgtgcccagctctcagaggcccttcttatgggactctcatatcctgaacctattatggccctgggaccccacagtgggaggcccatgaggcatcctggaaggcttctccttggcttctgcctgtggtacaaggcccctcctgtcccttaactatcctacccctcctactcttccatgctcctccttctcctcctgcactgctgcactcagcccccttctccccatcccctggccacccctgaccatcctcccttgctctttgtccttccccttttcttgcaggatgtggatgctgtggacaaggtgatgaaggagctggatgagaatggagatggggaggtggacttccaggagtatgtggtgctggtggctgccctgacagtggcctgcaacaacttcttctgggagaacagctgaagataacagccagggaggacaagcagggctgggcctagggacagactgcaagaggctcctgtcccctggggtctctgctgcatttgtgtcatcttgttgatggagttgtgatcatcccatctaagctagcttcctgtcaacacttctcacatcttatgctaactgtagataaagtggtttgatggtgacttcctcctctcccaccccatgcattaaattttaaggtagaacctcacctgttactgaaagtggtttgaaagtgaataaacttcagcaccatacagaagacaaatgcctgcttggtgtgctttctttcttcttgggaagatggtctgctagctctagaggatcccctagtgatggagttggccactccctctctgcgcgctcgctcgctcactgaggcctcctaggcaaagcctaggagtctggatacctttggtatccaggcctcagtgagcgagcgagcgcgcagagagggactggccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 13 - DNA, 2235 bp, synthetic construct
  misc_feature 1..2235: note = Synthetic sequence (S100A1+intron+ANF)
  source 1..2235: mol_type = other DNA
gaattctgtacagtagcttccaccatgggctctgagctggagacagctatggagaccctgatcaatgtgttccatgcccactctggcaaggagggagacaagtacaagctgagcaagaaggagctgaaggagctgctccagacagagctgtctggcttcctggatgcccagaaggtgagtgccatgctgtccccagcccagcatctccatctcacactcctcctggttcactgggtcctggtaatactggtccctgggtccctgtccctattccccaacagcccccttcagctcctgcatctgcccctgctgcctggccttcagcagtgttccatgtccacccattgaccactgcttgctggaagtgtctgagagctcctggggctgagcagagacactttcctggtgttccaaccctgggggtctccaacacttgaggcagcagctcagtgatctgagctggttacaaggacctggatgcaccaagccaaggacccccagtggaaggggagtgctgccaacagagaggtgcttctccccacatacacccaaggtcctggtgtgggcacaattaggctgagcctcaagctcacagtctttggagtcctatgtgcactaatgagggtcttaggtgaacagacactggcaaggaaatggcttagaggacactgatgctgcataccatgagcttagacctgggcccagtccttccttacccaccacccccagcccctggtccctagggcctctttgctacctaagggaagaacttcagcttcccctggaaggtctcctttgctgctcctgccaaaccactcctccctgggcaagaagcccctgctgggctggcttggctagggtactcccacctcccaataggagagaggctgtattgcctggtgacagtggcatggactttggagccataatgcctgggttgaatttctacctgtgcccctcactggctgtgtgacattggcagagttagtccactgttccttgcctccatttccacatggtaacactacaatatacttcagagggtgattgtgcagggttacagagataatactaattgttattattgctatagtgttccaaccactgttccaagcatgtcccatgtattaacttactatgccctcatagcagccctatgggttcatatctgggaaggtgctcagaagagcctggcacccactaaatgctcagcatgtcagccattgttatggcctctctagtcctgtgccttccacttttttctctttttttggttccacactgaactctgcaccaagccaaaggacacagatttgccaaactttggggcagcacctgggtggtgcatggggatgctactgctcaaagggcacagcttcctgggatggtgggcagctgggcatgggtgccccagaggggtctggggctgggctgctaggagggctccatgacacagcctccagctttgtgcccagctctcagaggcccttcttatgggactctcatatcctgaacctattatggccctgggaccccacagtgggaggcccatgaggcatcctggaaggcttctccttggcttctgcctgtggtacaaggcccctcctgtcccttaactatcctacccctcctactcttccatgctcctccttctcctcctgcactgctgcactcagcccccttctccccatcccctggccacccctgaccatcctcccttgctctttgtccttccccttttcttgcaggatgtggatgctgtggacaaggtgatgaaggagctggatgagaatggagatggggaggtggacttccaggagtatgtggtgctggtggctgccctgacagtggcctgcaacaacttcttctgggagaacagctgaagataacagccagggaggacaagcagggctgggcctagggacagactgcaagaggctcctgtcccctggggtctctgctgcatttgtgtcatcttgttgatggagttgtgatcatcccatctaagctagcttcctgtcaacacttctcacatcttatgctaactgtagataaagtggtttgatggtgacttcctcctctcccaccccatgcattaaattttaaggtagaacctcacctgttactgaaagtggtttgaaagtgaataaacttcagcaccatacagaagacaaatgcctgcttggtgtgctttctttcttcttgggaagatggtctgctagctctagaggatcc

SEQ ID NO: 14 - DNA, 1706 bp, Homo sapiens
  source 1..1706: mol_type = other DNA
gagaaggccaaggagctgtggcagagcatctataacttggaggcagagaagttcgacctgcaggagaagttcaagcagcagaaatatgaggtgggccgccatgctgtccccgcccagcatctccatctcacactcctcctggttcactgggtccggtaatactgctgcaggtccctgggtccctgtccctattccccaacagcccccttcagctcctgcatctgcccctgctgcctggccttcagcagtgttccatgtccaccgcattgacgactgcttgctggaagtgtctgagagctcgctggggctgagcagagacactttcctggtgttccaaccctgggggtctccaacacgttgaggcagcagctcagtgatctgagctggttacaaggaccaggatgcaccaagccaggacccccagtggaaggggagtgctgccaacagagaggtgcttctccccacatacacccaaggtcctggtgtgggcacaattaggctgagcctcaagctcacagtctttcggagtcctatgtgcactaatgagggtcttaggtgaacagacaccaggcaaggaaatggcttagaggacactgatgctgcataccggagcttagacctgggcgccagtccttccttacccaccacccccgagccccggtcccagggcctctttgctacctaagggaagaattcagctcctccctgggcaagaagcccctcgctgggcaggctcaggctgcagtggctacagggtactcccacctcccaatacggagagaggctgtattgcctggtgacagtggcatggactttggagccataatgcctgggttgaatttctacctgtgcccctcactggctgtgtgacattgggtgagttagtccactgttccttgcctccatttccacaggtaacactacaatatacttcagagggtgattgtgagggttacagagataatactaattgttattattgctatagtgttccaaccactgttccaagcatgtcccatgtattaacttactatgccctcatagcagccctatgggttcatatctgggaaggtgctcaggacagagcctggcacccactaaatgctcagcaggtgtcagccattgttatggcctctctagtcctgtgccttccacttttttctctttttttggttccacactgaactctgcaccggccaacaggacacagatttgccaaactttggggcagcaccctgcaggggtggtgcatggggatgctactgctcaaagggcacagcttccgggatggtgggcagctgggcaggggtgccccagaggggtctggggctgggctgctaggagggctccatgacacagcctccagctttgtgcccagctctcagaggcccttcttatgggactctcatatcctgaacctattatggccctgggaccccacagtgggaggcccatgaggcatcctggaagcttctccttggcttctgcctgtggtacacgggcccctcctgacccttaactatcctaacccctcctactcttccatgctcctccttctcctcctgcactgctgcactcagcccccttctccccatccccaggccacctgggacctgagccagtcagctccagcgttgctctttgtccttcccacttttcttgcagatcaatgttctccgaaacaggatcaacgataaccagaaagt